(19) United States
(12) Patent Application Publication
(10) Pub. No.: US 2019/0284279 A1
(43) Pub. Date: Sep. 19, 2019

(54) BISPECIFIC ANTIBODY BINDING TO HUMAN CD19 AND CD3

(71) Applicant: Beijing Luzhu Biotechnology Co., Ltd, Beijing (CN)

(72) Inventors: Jian KONG, Beijing (CN); Yi YE, Beijing (CN); Peng ZHOU, Beijing (CN); Ying HUANG, Beijing (CN); Qian KONG, Beijing (CN); Shuai YANG, Beijing (CN); Leitao XU, Beijing (CN); Kun ZHANG, Beijing (CN); Kaili ZHANG, Beijing (CN); Sisi WANG, Beijing (CN)

(73) Assignee: Beijing Luzhu Biotechnology Co., Ltd, Beijing (CN)

(21) Appl. No.: 16/237,124

(22) Filed: Dec. 31, 2018

Related U.S. Application Data
(63) Continuation of application No. PCT/CN2018/085397, filed on May 3, 2018.

(30) Foreign Application Priority Data
Nov. 15, 2017 (CN) 201711131955.0

(51) Int. Cl. C07K 16/28; A61K 47/65; C12N 15/85; A61K 9/00

(52) U.S. Cl. CPC C07K 16/2809; A61K 47/65; C12N 15/85; A61K 9/0019; C07K 2317/31; C07K 2317/92; C12N 2015/8518; C07K 2317/55; C07K 2317/622; C07K 2317/73; C07K 2317/522

(57) ABSTRACT

The present invention relates to a bispecific antibody that binds to human CD19 and CD3, wherein the bispecific antibody is composed of a Fab fragment which specifically recognizes a cell membrane antigen and a single-chain antibody which recognizes a CD3 molecule, where the single-chain antibody which recognizes the CD3 molecule is linked to the C-terminus of the CH1-region peptide fragment of the Fab fragment through a hydrophilic linker peptide-linker; and where the Fab fragment which specifically recognizes a cell membrane antigen contains a Fab structure which specifically recognizes a human CD19 antigen, and the bispecific antibody has the following structure:

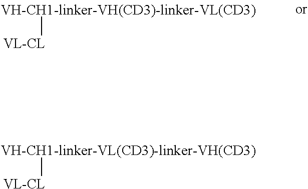

where the linker peptide-linker is composed of 8-20 hydrophilic amino acids.

This application is a continuation application of International Patent Application Serial No. PCT/CN2018/085397, filed 3 May 2018, entitled “BISPECIFIC ANTIBODY BINDING TO HUMAN CD19 AND CD3”, which itself claims priority to Chinese patent application 201711131955.0, filed 15 Nov. 2017. The aforementioned applications are hereby incorporated herein by reference and priority is hereby claimed.

### TECHNICAL FIELD

The present invention relates to the field of biotechnologies, and in particular to preparation of an antibody which is a bispecific antibody that binds to human CD19 and CD3.

### BACKGROUND

In the past 20 years, with the continuous improvement of human living standards and sanitary conditions, the life expectancy of human beings has increased continually, and the number of patients with malignant tumors has also increasingly grown. In fact, malignant tumors have become main diseases that seriously threaten human health. In recent years, various immunotherapy methods and drugs have developed rapidly, and significant progress has been made in various aspects such as antibodies, immunomodulators, and cell therapy. Tumor immunotherapy which regards T lymphocytes as main effector cells (such as chimeric antigen receptor T cells (CAR-T) and immune checkpoint inhibitors) has become a major breakthrough in the field of tumor immunotherapy.

Currently, there are only two bispecific antibody products approved for marketing by the governments worldwide, where one is catumaxomab developed by Trion Pharma, which can target a tumor surface antigen EpCAM (Epithelial cell adhesion molecule) and a T cell surface receptor CD3; and the other is blinatumomab (MT103) developed by Micromet and Amgen, which can simultaneously bind to CD19 and CD3. Both the two bispecific antibody products achieve the goal of treating tumors by activating and recruiting killer T cells. Catumaxomab belongs to a Triomab technology platform and consists of a tumor-targeting mouse IgG2a and a human CD3ε-targeting rat IgG2b, and meanwhile can also activate monocytes, macrophages, stellate cells and NK cells via a Fcγ receptor, thereby achieving “triple function” antibody activity. Due to the rare mismatch between the light and heavy chains of mice and rats, hybridomas respectively expressing mouse and rat antibodies were subjected to secondary fusion through hybridization of the hybridomas so as to obtain a hybridoma cell strain which simultaneously secretes a Triomab bispecific antibody and mouse and rat monoclonal antibodies. The mouse and rat monoclonal antibodies were then removed by affinity purification. Although catumaxomab is the first bispecific antibody approved for marketing, it has obvious limitations, which are mainly reflected in that, compared with a genetically engineered antibody, the Triomab antibody technology has a complicated production process and is difficult to control, and has the problem that this heterologous antibody easily causes immunogenicity. Blinatumomab is a bispecific antibody based on a Bispecific T cell Engager (BiTE) technology, which is composed of 2 single-chain variable fragments (ScFvs) containing only variable regions. Unlike the Triomab antibody, the BiTE antibody can be produced by large-scale culture of recombinant Chinese hamster ovary (CHO) cells, and the BiTE antibody contains only two binding domains, where one targets to a cancer cell surface antigen (e.g., CD19) with higher affinity, and the other targets to CD3 with lower affinity, and clinical trials have demonstrated that blinatumomab can still effectively activate T cells and remove tumor cells even at a very low dose. In July 2017, the US FDA approved blinatumomab constructed based on the molecular structure of a murine single-chain antibody for the treatment of B-cell lymphoma, and thus there has been zero breakthrough in using the genetically engineered bispecific antibody for immunotherapy of malignant tumors.

B-cell lymphoma is a solid tumor occurring in B cells of the blood system. It includes Hodgkin's lymphoma and non-Hodgkin's lymphoma (NHL), and has many classifications, where classic Hodgkin's lymphoma and nodular lymphocytes are the predominant Hodgkin's lymphoma, and are now considered as tumors originating from B cells. Five B-cell non-Hodgkin's lymphomas, i.e., diffuse large B cell lymphoma, follicular lymphoma, mucosa-associated lymphoid tissue lymphoma (MALT), small lymphocytic lymphoma/chronic lymphocytic leukemia, and mantle cell lymphoma (MCL), are most common, accounting for about ¾ of non-Hodgkin's lymphomas. Non-Hodgkin's lymphoma is a group of malignant tumors that originate in lymphoid tissues and diffuse throughout the body. Its morbidity and mortality have ranked fifth in malignant tumors, and most of the NHLs are derived from B lymphocytes (B lymphocyte non-Hodgkin's lymphomas, B-NHLs).

Markers on the surface of B lymphocytes have been widely recognized as targets for autoimmune diseases such as B cell lymphomas and B cell disorders. Markers present on surfaces of B lymphocytes are CD10, CD19, CD20, CD21, CD22, CD23, CD24, CD37, CD53, CD72, CD74, CD75, CD77, CD79a, CD79b, CD81-CD86, etc. Currently, monoclonal antibody drugs, which are targeted to molecules (such as CD20 and CD19) with high expression quantities on the surfaces of B lymphocytes, have been developed for the treatment of autoimmune diseases such as B cell lymphoma, rheumatoid arthritis and systemic lupus erythematosus. In particular, the anti-human CD20 monoclonal antibody (rituximab and the like) has become the drug of choice for the treatment of non-Hodgkin's lymphoma and is the most widely used worldwide.

It is well known that acute lymphoblastic leukemia (ALL) and many other B lymphocyte malignancies do not express CD20, or express CD20 at a low level, and only about half of patients with non-Hodgkin's lymphoma respond to CD20-controlled immunotherapy. CD19 is an important membrane antigen involved in B lymphocyte differentiation, activation, proliferation and antibody production, and is the best marker for diagnosing B lymphocyte tumors (leukemia, lymphoma) and identifying B lymphocytes. CD19 is a surface-specific marker of B lymphocytes and belongs to members of the immunoglobulin superfamily; is involved in B cell activation and signal transduction; is expressed in pre-B lymphocytes, immature B lymphocytes, mature B lymphocytes, and activated B lymphocytes; and has no expression in lymphoid pluripotent stem cells and other tissues. Most of the NHLs originate from B lymphocytes, more than 95% of B cell NHLs express a CD19 antigen, the CD19 antigen is more exposed, and there is no free CD19 in human serum; and thus, CD19 can be used as a target for the treatment of B cell lymphoma.

The CD19 molecule is a B-cell surface marker existing broader than CD20, is a receptor expressed on the surfaces of B cells, belongs to the immunoglobulin superfamily, and has the following ligands and related molecules: Complement receptor 2 (CR2, CD21), target of the antiproliferative antibody (TAPA-1, CD81), Leu-13, PI-3K, Vav, lyn and fyn. CD19 is an important signaling molecule that regulates the growth, activation and differentiation of B lymphocytes. CD19 can regulate signaling responses and plays an important role in regulating the signal threshold of B lymphocyte antigen receptors or other surface receptors. CD19 is a pan-B cell membrane glycoprotein that is differentiated and expressed from the early stage to the final stage during the development of a pre-B cell and can regulate the development and functions of B lymphocytes. The expression of CD19 is identified on most lymphoid-derived tumors, most non-Hodgkin's lymphomas (NHLs) and leukemias including chronic lymphocytic leukemias (CLLs), acute lymphoblastic leukemias (ALLs), and Waldenstrom's macroglobulinemias (WMs).

The expression of CD19 occurs throughout the life cycle of a B lymphocyte, in a primitive B cell, a pre-B cell, an early-developing B cell, a mature B cell, a plasma cell developed from the mature B cell, and a malignant B lymphoma cell. Most B lymphocyte-derived tumor cells such as pre-B acute lymphoblastic leukemias, chronic B lymphocytic leukemias, prolymphocytic leukemias, non-Hodgkin's lymphomas, hairy cell leukemias, common acute lymphocytic type leukemias and some non-acute lymphoblastic leukemias, multiple myelomas, plasmacytomas, etc., all express CD19 molecules.

CD3 is a marker present on the surfaces of all T lymphocytes. CD3 is also referred to as T3 or Leu-4. There are three subtypes, CD 3δ, CD 3ε and CD 3γ, respectively. The molecular weight of each of CD 3δ and CD 3ε is 20 kDa, and the molecular weight of CD 3γ is 26 kDa. CD3 is expressed on the surfaces of T lymphocytes, thymocytes and NK cytomembranes. It has 61%-85% expression in normal peripheral blood lymphocytes, and has 60%-85% expression in thymocytes. It belongs to the immunoglobulin superfamily. CD3 is part of a T lymphocyte receptor (TCR) complex, which forms the complex together with T cell receptors (TCRs) of α/β and γ/δ types, and is a major membrane antigen for signaling a peptide/MHC-bound TCR signal. TCR is essential for cell surface expression, antigen recognition, and signaling.

CD3 is a surface-specific molecule of T lymphocytes, through which T lymphocytes with killing effects can be recruited. Monoclonal antibodies against CD3 can induce or prevent activation of T lymphocytes. An anti-CD3 antibody can induce apoptosis of T lymphocytes in the presence of an anti-CD28 antibody or Interleukin-2 (IL-2). CD3 is one of the best markers of mature T lymphocytes in peripheral blood, and determining CD3+T lymphocytes plays an important role in classification diagnosis for evaluating immune deficiency (T-lymphocyte deficiency), leukemias, and lymphomas (of T lymphoblastic type). The anti-CD3 monoclonal antibody can be used for immunosuppressive therapy during organ transplantation or bone marrow transplantation, and can also be used for immunomodulatory treatment of severe autoimmune diseases to remove T lymphocytes. U.S. Pat. No. 4,361,549 describes a murine hybrid cell line for producing a monoclonal antibody OKT3 against antigens discovered in normal human T cells and cutaneous T lymphoma cells, and additionally, U.S. Pat. No. 5,885,573 describes a humanized monoclonal antibody constructed by transferring the murine OKT3 into a human antibody framework so to reduce the immunogenicity when the antibody is applied in the human body and reduce the occurrence rate of human anti-mouse antibody (HAMA) responses. OKT3 was the first murine monoclonal drug approved by the U.S. FDA in 1986 for the treatment of acute rejection of organ transplants, and was also the first monoclonal antibody drug approved by a government drug regulatory authority in the world. The main drawback of treatment using the murine OKT3 monoclonal antibody is T cell activation and HAMA responses caused by cytokine release due to cross-linking between T cells and FcγR-bearing cells. OKT3 has been used on the market for more than 10 years, and is finally replaced by humanized antibodies and new small-molecule immunosuppressive agents. On the other hand, OKT3 or other anti-CD3 antibodies can be used as immunopotentiators for stimulating T cell activation and proliferation. In in vitro cell culture, the anti-CD3 monoclonal antibody is used in combination with an anti-CD28 antibody or interleukin-2 to induce T cell proliferation. OKT3 is further used alone or as part of a bispecific antibody to target a cytotoxic T cell to tumor cells and virus-infected cells. So far, the method of using an antibody as a reagent for recruiting T cells has been hampered by several discoveries. Firstly, a natural or engineered antibody with high-affinity to T cells often does not activate the T cells to which it is bound; and secondly, a natural or engineered antibody with low affinity to T cells often performs poorly or is ineffective in inducing T cell-mediated lysis ability, such that it is important to select an anti-CD3 monoclonal antibody with the appropriate affinity.

Currently at home and abroad, inclusion of a bispecific antibody molecule specific for human CD19 and CD3 in an animal model and/or some limited clinical trial studies has shown significant effects. According to differences in the expression system platform technology as used, the effects are greatly different; in most of the published scientific literatures and patent documents, a prokaryotic expression system is used to express a small-molecule bispecific antibody, where such an expression system is fast and simple in operation, but the obtained bispecific antibody molecule always exhibits unsatisfactory effects, such as poor stability and is easy to lose its activity due to formation of a polymer. The problem of poor stability can only be solved by cryopreservating the bispecific antibody in an ultra-low temperature refrigerator or preparing the bispecific antibody into freeze-dried powder. A bifunctional antibody can be produced by methods such as hybrid hybridoma technology, connecting monoclonal antibodies through covalent coupling by chemical reactions, or genetic engineering techniques, where due to low bioactivity of some bifunctional antibodies and adoption of non-directional coupling techniques, the drug can exert certain effects only in a high dose. In many cases, it can be found in the stage of animal experimental models that these bifunctional antibodies obtained through covalent coupling by chemical reactions usually cannot exhibit obvious therapeutic effects.

Existing studies have shown that inclusion of a bispecific single-chain antibody molecule specific to human CD3 and human CD19 antigens [VL(CD19)-VH(CD19)-VH(CD3)-VL(CD3) is listed in Loffler, Blood 95 (2000), 2098-103; WO99/54440; Dreier, Int. J. Cancer. 100 (2002), 690-7, and patent WO99/54440] has a relatively accurate clinical effect, and it is also emphasized that the order of variable regions in the construct is not decisive. However, for a bispecific antibody for pharmaceutical use, mass production of drug molecules is possible only when researchers are able to provide reliable high-level expression and a viable purification technology route. It is most desirable that the designed and expressed protein drug molecule not contain non-essential peptide fragments, especially some peptide fragments that can produce antibodies in the human body. The C-terminus of the CD19×CD3 recombinant peptide chain listed in patent WO99/54440 and Chinese Patent No. 200480014513.2 carries a polyhistidine tail (His-Tag) consisting of 6 histidines (His), mainly for purification by means of metal ion chelation chromatography, and this tail consisting of 6 histidines is not removed from the final formed drug. The bispecific antibody blinatumomab with such a molecular structure was approved by the U.S. FDA in 2014 for the treatment of Philadelphia chromosome-negative precursor B-cell acute lymphoblastic leukemia.

The monoclonal antibody drug has been successfully used in the treatment of a variety of malignant tumors and human autoimmune diseases. Various monoclonal antibody drugs have excellent targeting properties and side effects which are significantly smaller than those of most chemical synthetic drugs. Although they are expensive, this does not stop their rapid growth trend. Under the present conditions that the living standards of people of countries all over the world are increasingly improved, numerous genetically engineered antibody drugs will also be favored by patients and medical personnel. At present, various monoclonal antibodies used clinically for treating malignant tumors are mostly of human IgG1 type, and exertion of their effects mainly depends on actions of antibody-dependent cellular cytotoxicity (ADCC) and complement dependent cytotoxicity (CDC). However, a monoclonal antibody of this structure has a large molecular weight; it is difficult for it to penetrate a tumor blood vessel to expert the killing effects it would have; and generally, the monoclonal antibody can reach a desired concentration and play an effective therapeutic role in the tumor only at a particularly high dose, and thus the treatment cost of the monoclonal antibody drug stays at a high level. Compared with a monoclonal antibody of an intact molecule, the micro-bifunctional antibody has the advantages of a small molecular weight and good tumor penetration, and has gradually become a focus of current research. This micro-bifunctional antibody drug molecule generally does not contain CH1, CH2, and CH3 structural regions of the human IgG antibody molecular structure, also does not contain an N-glycosylation site, and can be expressed using a non-mammalian cell expression system. There are also a variety of bifunctional antibodies with human intact IgG molecular structures, such as bispecific antibodies with intact CH1, CH2, and CH3 structures. Two antigen-binding (Fab) fragments of such a bifunctional antibody each recognize a specific antigenic site. The bispecific antibody of such a molecule structure generally requires interchange between the CH1 region and the CH of a light chain in a result design to increase the ratio of bifunctional antibody formation; or for example requires a single-chain antibody which recognizes the C-terminus connection of the heavy chain of an intact molecule of an antigen and also recognizes another antigen. The Fab fragment of a bifunctional antibody formed in such a way retains the affinity of the original antibody, while the affinity of ScFv at the C-terminal of the heavy chain of the bifunctional antibody is generally lower than that of the parent antibody.

Anti-tumor therapies mediated by a bispecific mini-antibody against the T cell surface antigen CD3 and relatively up-regulated antigens expressed on the surfaces of a malignant tumor cell membrane, tumor killing by relatively specific cytotoxic T lymphocyte (CTL) induced by in vitro sensitized dendritic cells, and the like, are considered to be the most promising adjuvant therapy means for removing residual tumor cells and micrometastases and thus radical curing tumors, in addition to traditional surgical treatments and radiotherapies and chemotherapies. Many animal experiments and clinical trials have confirmed the efficacy of biotherapy means. The bifunctional antibody has two antigen-binding arms, and can respectively bind to target cells and effector cells, so as to guide effector cells to target and kill tumors, thereby achieving targeted therapy of tumors.

### SUMMARY

The present invention provides a bispecific antibody having an asymmetric structure, which acts on human leukocyte differentiation antigens CD3 and CD19 as main targets, and through which the purpose of treating various B lymphocyte-derived malignant tumors by killing B cells with human body's own T cells is achieved. Unlike blinatumomab, the portion of the bispecific antibody of the present invention binding to a tumor antigen uses a structurally stable Fab, rather than a ScFv structure which is prone to polymerization, and has no 6×His tail at the C-terminus of a peptide chain.

The bispecific antibody of the present invention has two specific antigen binding sites, and thus it can simultaneously bind to a CD3 molecular complex on the surface of the CTL cell and a specific antigen on the surface of a target cell, without participation of a major histocompatibility complex (MHC)-class I molecule. The effect generated by such binding is significantly enlarged due to the presence of B7 on the surface of a B lymphocyte and the presence of a CD28 costimulatory molecule on the surface of a T cell, thereby activating the T cell to efficiently and accurately kill a tumor cell.

The bifunctional antibody of the present invention against human CD19 and CD3 has obvious advantages compared with blinatumomab already on the market. The portion of the bifunctional antibody against the human CD19 molecule (or other tumor molecules) is an intact humanized Fab antibody and has an affinity to human CD19 which is significantly better than that of a murine ScFv antibody, while the portion of the bifunctional antibody that binds to the human CD3 molecule adopts a ScFv molecule form with a weaker binding force, and the C-terminus of the bifunctional antibody does not contain a non-essential histidine tail. The bifunctional antibody of the present invention is a creative genetically engineered bifunctional antibody of a new generation, which uses an asymmetric structure form of Fab-ScFv. This new molecular form maintains the ability of binding to a tumor target antigen to the greatest extent, while appropriately weakening the capacity of binding to CD3 on a T cell. The purpose of selecting a relatively weaker CD3-binding force is to ensure that the cell signaling pathway can be activated only when the target and the CD3 are bound simultaneously. In absence of the target cell, the bifunctional antibody of the present invention does not have the effect of activating T cells at this concentration due to lack of action of essential costimulatory molecules.

To this end, the present invention provides a bispecific antibody that binds to human CD19 and CD3. The bispecific antibody is composed of a Fab fragment which specifically recognizes a cell membrane antigen and a single-chain antibody which recognizes a CD3 molecule, where the single-chain antibody which recognizes the CD3 molecule is linked to the C-terminus of the CH1-region peptide fragment of the Fab fragment through a hydrophilic linker peptide-linker;

The Fab fragment which specifically recognizes a cell membrane antigen contains a Fab structure which specifically recognizes a human CD19 antigen, and the bispecific antibody has the following structure:

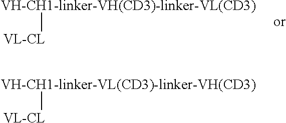

where the linker peptide-linker is composed of 8-20 hydrophilic amino acids.

Preferably, the bispecific antibody of the present invention has the following structure:

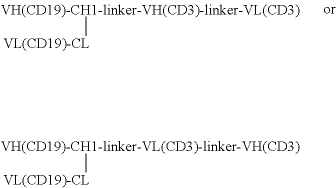

where the linker peptide-linker is a 2-3 fold polypeptide of the GGGGS form as a linker peptide.

In the following structures, the amino acid sequences of

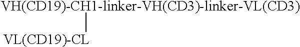

VH(CD19)-CH1-linker-VH(CD3)-linker-VL(CD3) are shown as 3 in the Sequence Listing,

the amino acid sequence of VH(CD19)-CH1-linker-VH(CD3)-linker-VL(CD3) is shown as 9 in the Sequence Listing,

the amino acid sequences of

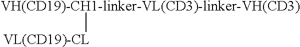

VH(CD19)-CH1-linker-VH(CD3)-linker-VL(CD3) are shown as 6 in the Sequence Listing,

and the amino acid sequence of VL(CD19)-CL is shown as 9 in the Sequence Listing,

where the nucleotide sequence contained in the heavy chain of a gene sequence containing the encoding leader peptide is shown as 1 in the Sequence Listing and corresponds to positions of the 14-70th nucleotides in the structural formula; the CD19 heavy chain variable region sequence and the human IgG CH1 sequence correspond to the positions of the 71-754th nucleotides; the linker peptide corresponds to the positions of the 755-799th and 1,175-1,219th nucleotides; VH(CD3) corresponds to the positions of the 800-1,174th nucleotides; and VL(CD3) corresponds to the positions of the 1,220-1,546th nucleotides.

The amino acid sequence contained in the heavy chain containing the leader peptide sequence is shown as 2 in the Sequence Listing; and the leader peptide sequence corresponds to the 1-19th amino acids. The 20-247th amino acids are VH (CD19)+IgG CH1; the 248-262th amino acids and the 388-402th amino acids are linker peptides (G4)₃; the 263-387th amino acids are VH(CD3); and the 403-511th amino acids are VL(CD3).

The nucleotide sequence contained in the heavy chain of a gene sequence containing the encoding leader peptide is shown as 4 in the Sequence Listing and corresponds to positions of the 14-70th nucleotides in the structural formula; the CD19 heavy chain variable region sequence and the human IgG CH1 sequence correspond to the positions of the 71-754th nucleotides; the linker peptide corresponds to the positions of the 755-799th and 1,127-1,171th nucleotides; VL(CD3) corresponds to the positions of the 800-1,126th nucleotides; and VH(CD3) corresponds to the positions of the 1,172-1,546th nucleotides.

The amino acid sequence contained in the heavy chain containing the leader peptide is shown as 5 in the Sequence Listing; where the 1-19th amino acids are a signal peptide sequence, the 20-247th amino acids are VH (CD19)+IgG CH1, the 248-262th amino acids and the 372-386th amino acids are linker peptides, the 263-371th amino acids are VL(CD3), and the 387-511th amino acids are VH(CD3).

The nucleotide sequence contained in the light chain containing the encoding leader peptide gene sequence is shown as 7 in the Sequence Listing, and corresponds to the positions of the 14-73th nucleotides.

The amino acid sequence of the light chain containing the leader peptide sequence is shown as 8 in the Sequence Listing, and corresponds to the positions of the 1-20th amino acids of the structural formula.

The sequence of amino acids contained in the heavy chain not containing the leader peptide is shown as 3 and 6 in the Sequence Listing.

The sequence of amino acids contained in the light chain not containing the leader peptide is shown as 9 in the Sequence Listing.

The single-chain antibody which recognizes the CD3 molecule has a structure in the form of ScFv, is targeted to human CD3a, and can be derived from the variable region gene sequences of various monoclonal antibodies currently known, including but not limited to CD3-specific antibodies OKT3, X35-3, WT31, WT32, SPv-T3b, TR-66, 11D8, 12F6, M-T301, SMC2 and F101.01.

The Fab structural fragment specifically recognizing the human CD19 antigen can be derived from the sequences of the light chain variable regions and heavy chain variable regions of various well-known murine anti-human CD19 monoclonal antibodies, such as 4G7, B43, CLB-CD19, 5J25-C1, Leu-12, HD37 or other known variable region sequences of a monoclonal antibody against human CD19, or the sequences of the light chain variable regions and heavy chain variable regions of a monoclonal antibody against human CD19 constructed by our company are used.

The present invention further provides a method for preparing the bispecific antibody of the present invention. The bispecific antibody is prepared by a genetic recombination technology, and can be expressed in a CHO cell using various forms of mammalian cell expression vectors, preferably using a GS expression system. CHO cells are cultured using a chemically defined medium, and no hormones or proteins of various toxic origins or hydrolyzates thereof are added during the culture.

The preparation method of the present invention includes linearizing a single plasmid vector containing the bispecific antibody gene by single endonuclease digestion; transfecting the linearized plasmid vector into a CHO cell to obtain a positive clone strain; culturing the positive clone strain in a bioreactor, such that the product is secreted into the supernatant of the culture solution; purifying by an ion exchange chromatography medium or affinity chromatography combined with ion exchange chromatography to obtain a bispecific antibody which can specifically bind to human CD19 and CD3.

The present invention further provides use of the bispecific antibody of the present invention in preparation of drugs for treating various human-B-cell-derived malignant tumors or immune disorders such as various B cell leukemias (lymphomas), non-Hodgkin's lymphomas, and serious autoimmune diseases such as rheumatoid arthritis and ankylosing spondylitis.

The present invention further provides a pharmaceutical composition including the bispecific antibody of the present invention. The pharmaceutical composition may be prepared into a liquid preparation or a freeze-dried preparation, and may be continuously administered by using a continuous infusion pump; or may be administered by a pulsed infusion pump at a fixed time, where intravenous administration is recommended; or may be administered by subcutaneous injection.

High level of stable expression and purification by liquid chromatography of the bifunctional antibody constructed by the technology of the present invention are successfully achieved in the CHO cell. A bifunctional antibody with extremely high purity can be obtained by the technique and method of the present invention, and a variety of liquid preparations of the antibody have been developed. By using the liquid preparation formulation of the present invention, the bispecific antibody of the present invention has a stable quality when stored in a concentration range of 0.1-10 mg/ml with protection from light at 2-8° C.

The present invention provides a tumor-targeting molecule antibody capable of producing high affinity and a CD3 antibody having relatively weak binding ability. The two antibodies are linked by a hydrophilic peptide chain of a suitable length to provide a sufficient space for free stretching of the antibody-specific binding moiety. This molecular structure has good thermal stability and less polymer formation, and ensures the stability and excellent binding ability of the antibody molecule to the greatest extent. The excellent liquid preparation formulation and stable molecular structure provides guarantee for convenient safe clinical administration.

The drug of the present invention is mainly used for the treatment and/or alleviation of B cell-associated or B-cell mediated disorders.

In preferred embodiments of the bispecific antibody of the present invention, the VH and VL regions of the CD3 specific domain may be derived from a variety of currently known monoclonal antibodies against the human CD3, such as CD3-specific antibodies like OKT3, X35-3, WT31, WT32, SPv-T3b, TR-66, 11D8, 12F6, M-T301, SMC2 and F101.01. The specificity of each of these CD3 monoclonal antibodies is known in the field of immunology research, and is described in various forms of publications. In a more preferred embodiment, the VH and VL regions of the CD3 specific domain are derived from OKT-3 or TR-66, or derived from derivatives of antibodies against CD3ε.

In a preferred embodiment of the present invention, the sequences of the humanized CD19 monoclonal antibody may be derived from the sequence of the light chain variable region and heavy chain variable region of a murine anti-human CD19 monoclonal antibody well-known to scientific researchers in the art, such as 4G7, B43, CLB-CD19, SJ25-C1, Leu-12, HD37 or other known variable region sequences of a monoclonal antibody against human CD19, or the sequences of the light chain variable regions and heavy chain variable regions of a monoclonal antibody K19 against human CD19 constructed by our company are used.

The anti-human-CD19-and-CD3 bispecific antibody molecule of the present invention is a genetically engineered antibody molecule capable of simultaneously binding to a CD19 molecule on the surface of a human B cell and a human CD3 molecule. The bispecific antibody molecule is constructed by: embedding a heavy chain variable region gene against the human CD19 molecule, a human IgG CH1 region gene, a hydrophilic linker peptide gene, and an anti-human CD3 single-chain antibody into an expression vector, and then embedding a gene containing the light chain of the anti-human CD19 monoclonal antibody (containing the CL region of a kappa chain) into the expression vector, such that the constructed plasmid vector contains a gene fully expressing the anti-human CD19 monoclonal antibody Fab and the anti-human CD3ε single-chain antibody; digesting the plasmid with a endonuclease for linearization; then transfecting the linearized plasmid into a CHO cell by an electrotransfection instrument; screening out a positive clone expressing anti-human CD19 and CD3ε; further screening out a positive clone with high expression under pressure; establishing a cell seed bank, and storing the cell seed bank in a liquid nitrogen container. The binding activities of the expressed bispecific antibody to human T lymphoma cells and human B lymphoma cells are determined by a flow cytometer. The obtained high expressing clone strains are cultured in a chemically defined medium for a certain period of time, then centrifuged to collect the supernatant, purified through multiple times of purification processes by ion exchange chromatography to obtain a bispecific antibody having a monomer purity greater than 98%. In the presence of the B cell and the T cell, the bispecific antibody can continually and efficiently activate T lymphocytes to kill B lymphocytes. For the bispecific antibody of the present invention, the nucleotide sequence of the heavy chain containing the leader peptide gene is shown as SEQ ID No.1, the nucleotide sequence of the light chain containing the leader peptide gene is shown as SEQ ID No. 2, the amino acid sequence of the heavy chain containing the leader peptide is shown as SEQ ID No. 3, and the amino acid sequence of the light chain containing the leader peptide is shown as SEQ ID No. 4. The nucleotide sequence and amino acid sequence of the heavy chain VH(CD19)-CH1-linker-ScFv(CD3ε) without the leader peptide are shown as SEQ ID No. 5 and SEQ ID No. 7, and the nucleotide sequence and amino acid sequence of the light chain VL(CD19)-CH1 of the CD19 antibody without the leader peptide are shown as SEQ ID No. 6 and SEQ ID No. 8.

The heavy chain of the bispecific antibody of the present invention contains a segment of hydrophilic polypeptide which links the CH1 region in the heavy chain of the anti-human CD19 monoclonal antibody Fab to an anti-human CD3 single chain antibody, where in order to provide the CD3-specific single-chain antibody with a larger degree of freedom, the length of the linker peptide should be no shorter than 8 amino acids and no longer than 20 amino acids; the length of the amino acid peptide chain is preferably an integral multiple of the GGGGS form commonly used in the industry, or a non-integral multiple thereof, where preferably 2-3 times of the GGGGS form is used as a linker peptide, and that is, a length of the linker peptide can be between 10 to 15 among amino acids is a preferred length.

In an embodiment of the present invention, a specific method for producing the drug of the present invention is provided, which includes construction of an asymmetric bispecific antibody vector as defined herein, plasmid transfection, cell strain cloning, bioreactor culture assay, purification of bifunctional antibody, stable liquid preparation formulation and a stability test therefor, a T lymphocyte proliferation test, a B lymphocyte killing test, a tumor-bearing mouse model test, and the like, and the corresponding methods are described in preferred embodiments.

The bispecific antibody of the present invention can be used for the treatment of various malignant tumors derived from B cells, and can also be used for the treatment of autoimmune diseases and B cell-derived immune disorders.

Hereinafter, we will introduce the bispecific antibody of the present invention and the use thereof by way of embodiments.

### BRIEF DESCRIPTION OF THE DRAWING

FIG. 1 shows an electrophoresis photograph of an enzyme-digested 193HVkP recombinant plasmid, where
Lanes 1-4: Enzyme-digested product of plasmid 193HVkP
Lane 5: DL2000 (2000 bp, 1000 bp, 750 bp, 500 bp, 250 bp, 100 bp)
Lane 6: DL10000 (10000 bp, 7000 bp, 4000 bp, 2000 bp, 1000 bp, 500 bp, 250 bp).

FIG. 2 shows a non-reducing SDS-PAGE analysis result of the CD19-CD3 bispecific antibody.

FIG. 3 shows a reducing SDS-PAGE analysis result of the CD19-CD3 bispecific antibody.

FIG. 4 shows an HPLC-SEC chromatogram of the CD19-CD3-bispecific antibody.

FIG. 5 shows a reducing or non-reducing CE-SDS electrophoretogram of the K193 antibody.

FIG. 6 shows an LC-MS mass spectrogram of the CD19-CD3 bispecific antibody.

FIG. 7 shows an LC-MS mass spectrogram of the CD19-CD3 bispecific antibody.

FIG. 8 shows binding reactions of the CD19-CD3 bispecific antibody (upper) and OKT3 (lower) with a Jurkat cell.

FIG. 9 shows reaction between the CD19-CD3 bispecific antibody and a CD19 positive cell (flow cytometer).

FIG. 10 shows binding reaction between the CD19-CD bispecific antibody and a CD19 positive B cell.

FIG. 11 shows reactions of the CD19-CD3 bispecific antibody and the CD monoclonal antibody K19 with the Raji cell (flow cytometer).

FIG. 12 shows reactions of the CD19-CD3 bispecific antibody and the CD monoclonal antibody K19 with the Raji cell (flow cytometer).

FIG. 13 shows reactions of the CD19-CD3 bispecific antibody and the CD monoclonal antibody K19 with the Raji cell (flow cytometer).

FIG. 14 shows reactions of the CD19-CD3 bispecific antibody and the CD19 monoclonal antibody with the Raji cell (flow cytometer).

FIG. 15 shows a dose response curve of the K193 antibody activating T cell to kill B cells.

FIG. 16 shows a curve of a specific binding reaction between the K193 antibody and a recombinant human CD3ε.

FIG. 17 shows a curve of a specific binding reaction between the K193 antibody and a recombinant human CD19.

FIG. 18 shows the effect of the existence of the B cell on the effects of the K193 antibody.

FIG. 19 shows the effects of a B7:CD28 costimulatory-molecule monoclonal antibody on K193-activated expression of CD69 in lymphocytes.

FIG. 20 shows the effects of a B7:CD28 costimulatory-molecule monoclonal antibody on K193-activated expression of CD25 in lymphocytes.

FIG. 21 shows a killing percentage of Raji cells by antibodies K193 and BLI193.

FIG. 22 shows a killing percentage of Namalwa cells by antibodies K193 and BLI193.

FIG. 23 shows a killing percentage of Daudi cells by antibodies K193 and BLI193.

FIG. 24 shows a killing percentage of K562 cells by antibodies K193 and BLI193.

### DETAILED DESCRIPTION

The present invention is further illustrated in connection with the following embodiments.

### Embodiment 1. Construction of Plasmid Expression Vector

Construction of Plasmid LZ19HT (pMD19-T Vector+CD19VH+hIgG1 CH1) and Construction of LZ19VkT(pMD19-T Vector+CD19Vk+hIgG1 Ck)

Primers H-F1, LZ19H-F2 and LZ19H-R1 were used to amplify a fragment CD19VH+hIgG1 CH1 from a plasmid PTYS-KJ2-h containing the heavy chain of a humanized anti-CD19 monoclonal antibody, and a KOZAK sequence, a heavy chain signal peptide sequence, a Linker ((G4S)3) and a restriction enzyme cut site were introduced into the fragment, then an A tail was added to the fragment, and the fragment was connected with a pMD19-T Vector to obtain a plasmid LZ19HT;

Primers P71-F1, LZ19Vk-F2 and LZ19Vk-R1 were used to amplify a CD19Vk+hCk gene fragment from a plasmid PTYS-KJ2-1 containing the light chain of a humanized anti-CD19 monoclonal antibody, a KOZAK sequence, a light-chain signal peptide sequence and a restriction enzyme cut site were introduced into the fragment, then a tail was added to the fragment, and the fragment was connected with a pMD19-T Vector to obtain a plasmid LZ19VkT;

Different clones of LZ19HT and LZ19VkT were sent to a sequencing company (Beijing Invitrogen Biotechnology Co., Ltd.) for sequencing, and exact clones were picked out for an experiment of the next step, where the serial numbers of the clones were LZ19HT36 and LZ19VkT20;

| No. of | |
|---|---|
| primer | primer sequence (5′→3′) |
| H-F1 | CCCAAGCTTAATTGCCGCCACCATGGAATGGAGCTGG |
| | GTGTTCCTGTTCTTTCTGTCC |
| LZ19H-F2 | TTCCTGTTCTTTCTGTCCGTGACCACAGGCGTGCATT |
| | CTCAGGTGCAGCTGCAGCAG |
| LZ19H-R1 | CGCCACCGCCGGATCCACCTCCGCC |
| P71-F1 | CCCAAGCTTAATTGCCGCCACCATGTCTGTGCCTACC |
| | CAGGTGCTGGGACTGCTGCTG |
| LZ19Vk-F2 | CTGGGACTGCTGCTGCTGTGGCTGACAGACGCCCGCT |
| | GTGACATCCAGCTGACACAGT |
| 19Vk-R1 | CCG GAATTC TCATTA GCTACACTCTCCCCTG |

Construction of Plasmid 19H3HVkP(pXC184+CD19VH+HIgG1 CH1+Anti-Human-CD3-ScFv) and Construction of 19VkP(pXC174+CD19Vk+hIgG1 Ck)

LZ19HT36, PXC-184 and LZI2CHL (Anti-Human-CD3-ScFv sequences) were processed by corresponding restriction endonucleases to obtain LZ19HT-HindIII/BamHI, LZI2CHL-BamHI/EcoRI and pXC18.4-HindIII/EcoRI. The three enzyme-digested products were ligated, transformed and screened to obtain a positive clone 19H3HVkP(pXC184+CD19VH+HIgG1 CH1+Anti-Human-CD3-ScFv).

LZ19VkT20 and PXC-17.4 were processed by corresponding restriction endonucleases to obtain LZ19VkT-HindIII/EcoRI and pXC174-HindIII/EcoRI. The 2 enzyme-digested products were ligated, transformed and screened to obtain a positive clone 19VkP(pXC174+CD19Vk+hIgG1 Ck).

### Construction of 193HVkP

19H3HVkP and 19VkP were processed by restriction endonucleases NotI and PvuI to obtain enzyme-digested products 19H3HVkP-N/P and 19VkP-N/P. The two enzyme-digested products were ligated by a ligase, transformed and screened to obtain a positive clone 193HVkP. Plasmids 193HVkP were extracted in large amount, linearized by a restriction endonuclease PvuI, and purified through phenol extraction to obtain linearized plasmids 193HVkP-straight; and the agarose electrophoresis photograph of the plasmid was shown in FIG. 1.

### Embodiment 2. Establishing and Screening of Stable Cloned Strains

In a sterile lamiar flow bench, a perforation voltage of a gene pulse generator Xcell (Bio-Rad) was set as 300V, 900 μF, and exponential pulse, where a disposable electric shock cup with a gap of 4 mm was taken out, added with 40 μg linearized plasmid DNA (100 μl) and 0.7 ml of a suspension of CHO K1 cells (GS KO); the voltage of the electroporation apparatus was set to infinity, and the linearized plasmid 193HVkP-straight was directly transfected into a CHO K1 cell by electrotransfection; the cells in the electric shock cup were transferred into a triangular culture flask, added with a CD CHO culture solution, and cultured on a shaker under 5% CO₂ at 36-37° C. and 135 rpm for 24 h; the cells were collected by low speed centrifugation, and the culture solution was replaced by a CD CHO culture solution containing 50 μM MSX (without glutamine), monoclonal cell strains were obtained by a limiting dilution method, then a clonal strain with a higher expression level was selected through a ELISA method (murine monoclonal antibodies against human κ chain+expression products K193+goat anti-human IgG-HRP) and subcultured to finally obtain 6 clonal strains with the clone serial numbers of 45610, 45D10, 41C11, 4166, 45C7 and 45F6 respectively; the binding activity of each clonal-strain expression product to Jurkat cells and Raji cells was detected by a flow cytometer; and according to the expression amount of antibodies in the cultural supernatant and the result of the binding activity test, a 41C11 clonal strain (K193) was selected for a amplification test.

### Embodiment 3. Purification of Expression Products of the CD19-CD3 Bispecific Antibody

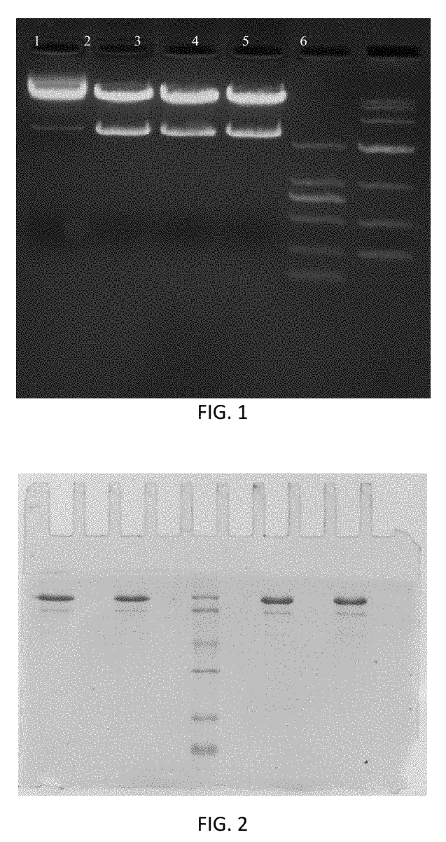

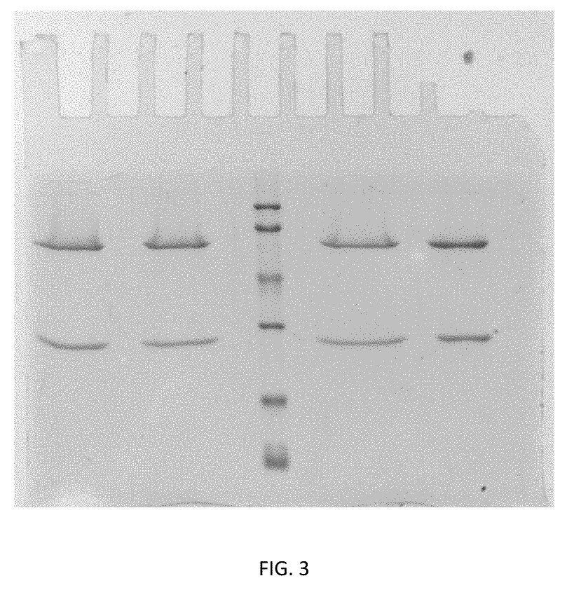

The obtained 41C11 clonal strain (K193) was inoculated into a 2 L triangular flask containing 500 ml CD CHO culture solution followed by tightening of a venting cap, and then cultured on a shaker under 5% CO₂ at 36-37° C. and 135 rpm for 7 days; when the density of viable cells was dropped to between 60%-70%, high speed centrifugation was conducted at 12,000 r/min (revolutions per minute) to remove cells and cell debris, and the supernatant of the cell culture was collected and then limitedly diluted with pure water or a 20 mM citrate phosphate buffering solution at pH 5.0-6.0 until the electrical conductivity of the solution was no more than 4 mSiemens/cm; then the solution flowed through a chromatographic column packed with strong cation exchange gel Eshmuno S (XK26*20 cm, GE HealthCare), where under this condition, the Eshmuno S gel can adsorb the CD19-CD3 bispecific antibody; after completion of sample loading, the column was washed with the citrate phosphate buffering solution to the baseline, and the concentration of sodium chloride was increased by gradient to sequence elute the protein bound onto the gel, where the earliest peaking one was the anti-CD19-CD3 antibody. After the buffering solution was subjected to ultrafiltration substitution, the solution was purified through a POROS XQ strong-anion-exchange chromatography column to harvest CD19-CD3 bispecific antibodies, and then the purified CD19-CD3 bispecific antibodies were subjected to electrophoresis with 12.5% SDS-PAGE (Mini-PROTEAN Tetra System, Bio-Rad). When the bromophenol blue indicated that the antibodies was electrophoresed to the lower end of the gel glass plate, the electrophoresis was stopped, and the gel was taken out, stained with Coomassie blue staining solution for 2 h, decolorized until the background was bright and photographed. The electrophoresis results are shown in FIGS. 2 and 3.

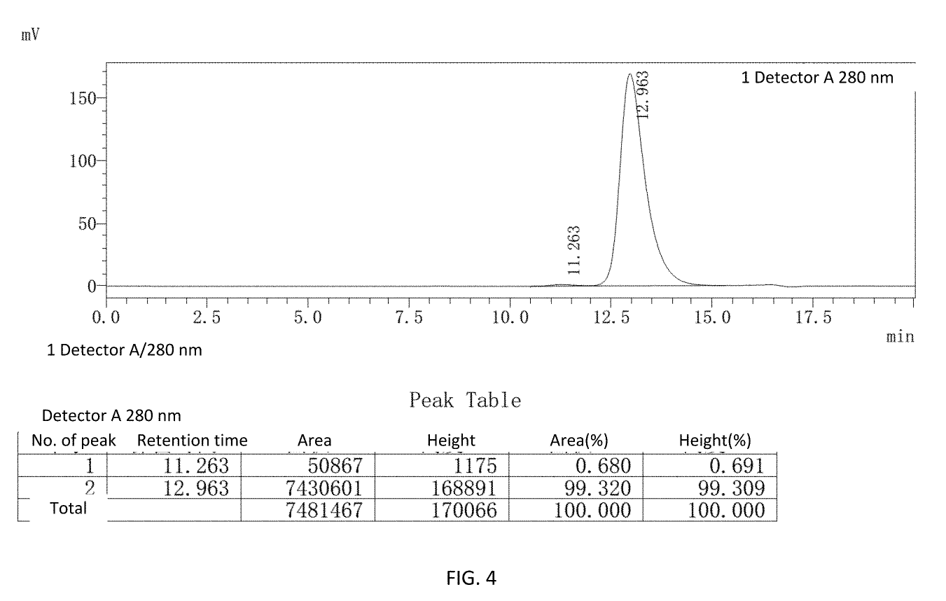

The purified CD19-CD3 bispecific antibody was subjected to monomer and polymer content analysis on a Shimadzu LC-20AT HPLC TSK 3000SWxl (7.8×300 mm) analytical column with 40 mM phosphate buffer saline (PBS) (containing 0.5M Na₂SO₄, pH 6.5) as mobile phase. The detection results showed that the High Performance Liquid Chromatography (HPLC) purity was not less than 95%, the polymer content was less than 5%, and no visible impurity peak was observed. The results are shown in FIG. 4.

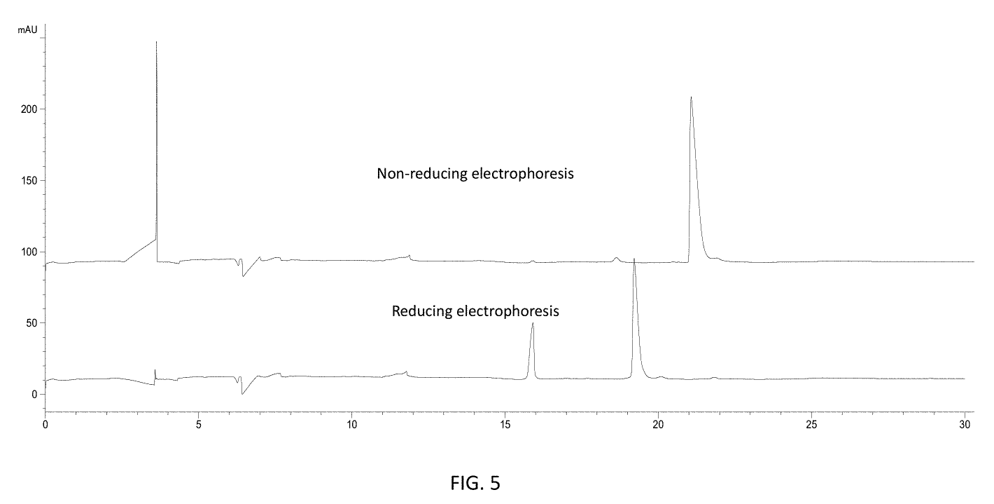

We performed reducing and non-reducing Capillary Electrophoresis-Sodium Dodecyl Sulfate (CE-SDS) analysis of the purified CD19-CD3 bispecific antibody on an Agilent CE 7100 high performance capillary electrophoresis instrument. Using an uncoated molten capillary column (with an inner diameter of 50 μm, a total length of 33 cm, an effective length of 24.5 cm), under reducing and non-reducing conditions, the quantitative determination of the purity of the CD19-CD3 bispecific antibody (K193 antibody) was conducted according to a molecular weight. 86 μl K193 antibodies desalted by ultrafiltration (with a protein content of 1 mg/ml) was taken, added with 9 μL sample buffer (100 mM Tris-HCl containing 1% SDS, pH 8.3), added with 5 μL β-mercaptoethanol/5 μl 250 mM iodoacetamide, well mixed and placed in a water bath at 70° C. for 10 min to obtain a reduced/non-reduced electrophoresis sample. CE-SDS parameters: electrokinetic injection, injection at −5 kV for 80 s; a separation voltage of −16.5 kV, and a pressure rising time of 1 min; a DAD detection wavelength of 220 nm, and a bandwidth of 4 nm (Reference off); a sampling frequency set at 2.5 Hz; and an inlet and outlet buffer bottle pressure of 2 bar during operation. FIG. 5 is a reducing/non-reducing electrophorogram of the K193 antibody: the results indicate that the purity of the CD19-CD3 bispecific antibody exceeds more than 95%.

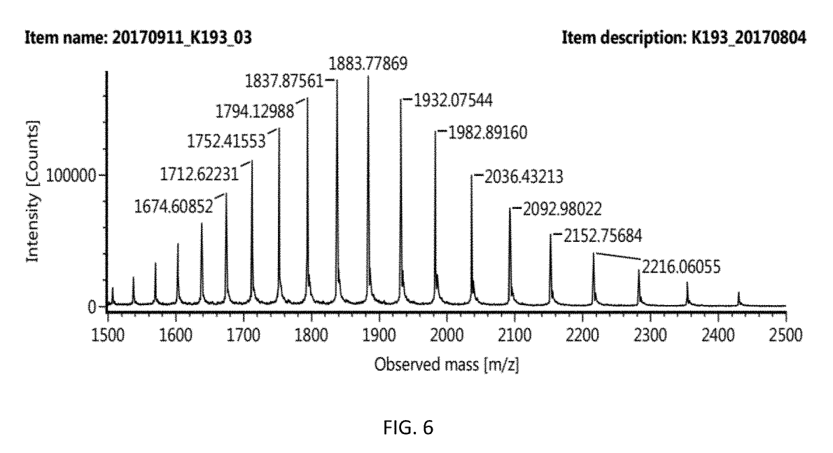

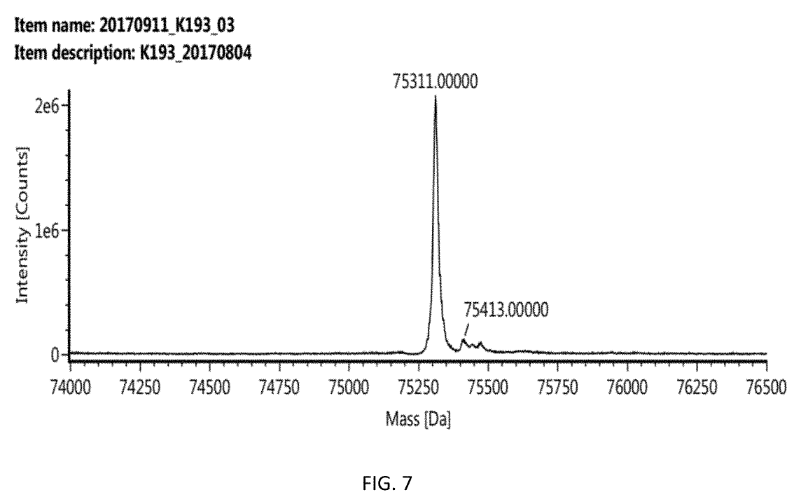

The intact protein molecular mass of the CD19-CD3 bispecific antibody (K193 antibody) was determined by using waters ACQUITY UPLC-Xevo G2-XS QT of liquid chromatography-mass spectrometry system (an ultra-performance liquid chromatography quaternary pump of bioQuaternary Solvent Manager type, an UV detector of TUV Detector type, an autosampler of bioSamples Manager-FTN type, and a Waters Xevo G2-XS Q Tof tandem quadrupole flight mass spectrometry system). Data acquisition was performed using MassLynx™ 4.1 software and the data was processed using UNIFI Portal software. UPLC parameters: mobile phase A: 0.1% aqueous formic acid; mobile phase B: 0.1% formic acid in acetonitrile; chromatographic column: XBridge Protein BEH C4, 2.1 mm×100 mm, 3.5 μm; flow rate: 0.300 mL/min; detection wavelength: 280 nm; sample concentration: 0.5 mg/ml; injection volume: 1 μL; column temperature: 80° C.; sample tray temperature: 10° C.; running time: 10 min; procedure: 0-1 min of 5% B, 6-7 min of 95% B, 7.5-8 min of 5-95% B, 8.5-9 min of 5-95% B, and 9.5-10 min of 5% B. Mass spectrometry parameters: ESI mode: positive ion MS(+), sensitivity mode; capillary voltage: 3 kV, cone voltage: 180 V, offset: 150 V; flow rate of desolvation gas (N2): 800 L/h, temperature of desolvation gas: 450° C., source temperature: 120° C.; and mass scanning range: 600-4,500. The data was processed using the UNIFI Portal software. The deconvolution parameters were: a m/z range of 1,500-2,500; an output molecular weight range of 70,000-80,000; and a manual peak width mode was selected, with a starting peak width of 0.1, and an end peak width of 0.2; and the maximum number of iterations was 18. The N-terminal pyroglutamate modification was selected as a variable modification. FIGS. 6 and 7 is a mass spectrogram and deconvolution map obtained from the molecular weight detection of the K193 antibody. The intact molecular weight of the CD19-CD3 bispecific antibody as detected by UPLC-ESI QTOF was 75,311.

### Embodiment 4. Measurement of Binding Activity of the CD19-CD3 Antibody to a CD3 Positive Cell by Flow Cytometer

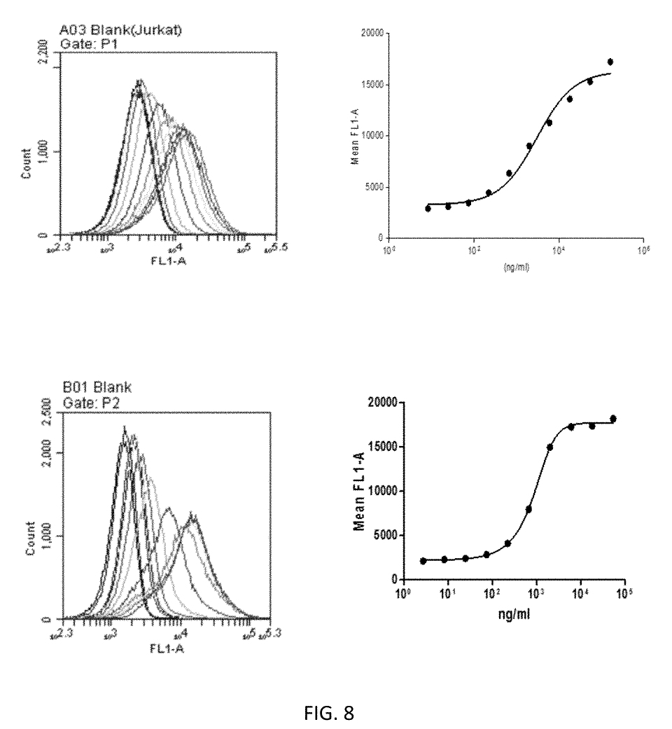

To test the binding ability of the CD19-CD3 antibody to CD3, we performed flow cytometry analysis (FACS) on the obtained bispecific antibody. The K193 antibody was diluted with 0.02 mol/L PBS (pH 7.4, containing 1% BSA) until the initial protein concentration was 162 μg/ml (the initial concentration of the control OKT3 monoclonal antibody was 54 μg/ml). A 96-well U-shaped plate was taken, and 50 μl of 0.02 mol/L PBS (pH 7.4, containing 1% BSA) was added into 10-12 wells of row A as a blank control wells. 50 μl of 0.02 mol/L PBS (pH 7.4, containing 1% BSA) was added into 2-10 wells of row B, and then 75 μl of the K193 antibodies with an antibody content diluted to 162 μg/ml was added into the well B1. 25 μl of the antibody solution in the well B1 was pipetted into the well B2, well mixed and then diluted sequentially until the well B10 in a 3 fold gradient, well mixed, and 25 μl of the solution was pipetted from each well and discarded to keep a volume of 50 μl per well. The dilution range was 162 μg/ml-0.0082 μg/ml, with 10 dilutions in total. A Jurkat cell suspension with a cell density of 5.0×10⁶ cells/ml was prepared, and 100 μl of the cell suspension was sequentially added into each of the aforementioned sample wells, well mixed, reacted at room temperature for 60 min, and centrifuged, and then the supernatant was carefully pipetted and discarded. Except that the blank control wells A10 and A12 were each added with 50 μl of 0.02 mol/L PBS (pH 7.4, containing 1% BSA), the remaining wells were each added with 50 μl of a mouse anti-human IgG K chain monoclonal antibody diluted to 2 μg/ml, well mixed and reacted at room temperature for 60 min, and centrifuged, and then the supernatant was carefully pipetted and discarded. The blank control wells A10 and A11 were each added with 50 μl of 0.02 mol/L PBS (pH 7.4, containing 1% BSA), and the sample wells were each added with 50 μl of the well diluted FITC-labeled goat anti-mouse IgG (1:1000), well mixed and reacted at room temperature with protection from light for 30 min, and centrifuged, and then the supernatant was carefully pipetted and discarded. 150 μl of 0.02 mol/L PBS (pH 7.4) was added into each well, and the cells in the well were resuspended. The flow cytometer in-door sample loading quantity was set as 50,000 events, and the flow rate was set as Fast. The cells were well mixed by carefully pipetting up and down them with the tip of a pipette, the cell suspension was transferred into a 0.5 ml centrifuge tube, and the cell fluorescence values were determined sequentially; and meanwhile by using a GraphPad Prism5.0 software, the EC50 value of the K193 antibody was calculated as 4.01×10⁻⁸ mole/L, and the EC50 value of the control OKT3 was calculated as 6.09×10⁻⁹ mole/L, the binding activity of the K193 antibody to CD3 was only about 1/10 of that of OKT3, and the binding ability of K193 to a CD3ε molecule on the surface of a T cell was significantly weaker than that of OKT3, and the results were shown in FIG. 8.

### Embodiment 5: Test of Binding Activity of the CD19-CD3 Bispecific Antibody to a CD19 Positive Cell

Raji cells, Daudi cells, and IM-9 cells are all B lymphoma cells, on the cell surface of which was each provided with a CD19 antigen that can specifically bind to the CD19 antigen on the cell surface. The specific binding conditions of the bispecific antibody sample K193 to the CD19 sites of the Raji cells, the Daudi cells and the IM-9 cells were detected by a flow cytometer. In this experiment, a CD19-negative K562 cell was used as a negative control.

1. Binding Reaction of the CD19-CD3 Bispecific Antibody to Various B Cell Lymphoma Cells

The specific binding activities of the K193 antibody to the CD19 sites of the Raji, Daudi, IM-9 and K562 cells were detected with a flow cytometer (Accuri™ C6 Flow Cytometer, Becton Dickinson). The K193 antibody was diluted with 0.02 mol/L PBS (pH 7.4, containing 1% BSA) until the initial protein concentration was 18 μg/ml. One 96-well U-shaped plate was taken, and 50 μl of 0.02 mol/L PBS (pH 7.4, containing 1% BSA) was added into 10-11 wells of row A as a blank control wells. 50 μl of 0.02 mol/L PBS (pH 7.4, containing 1% BSA) was added into 2-9 wells of each of rows B, C, D and E, and then 75 μl of the K193 antibodies with an antibody content diluted to 18 μg/ml was added into each of the wells B1, C1, D1 and E1. 25 μl of the antibody solution in the well of column 1 was pipetted into the well of column 2, well mixed and then diluted sequentially until the well of column 9 in a 3 fold gradient, well mixed, and 25 μl of the solution was pipetted from each well and discarded to keep a volume of 50 μl per well. The dilution range was 18 μg/ml-0.0027 μg/ml, with 9 dilutions in total. A cell suspension with a cell density of 5.0×10⁶ cells/ml was prepared, and 100 μl suspensions of Raji, Daudi, IM-9 and K562 cells were respectively added into the aforementioned sample wells in rows B, C, D and E, well mixed, reacted at room temperature for 60 min, and centrifuged, and then the supernatant was carefully pipetted and discarded.

| | Raji Mean FL1-A | Daudi Mean FL1-A | IM-9 Mean FL1-A | K562 Mean FL1-A |
|---|---|---|---|---|
| Antibody Content | (sample-negative | (sample-negative | (sample-negative | (sample-negative |
| (ng/ml) | control) | control) | control) | control) |
| 2.7 | 112.06 | 328.46 | 568.19 | 41.87 |
| 8.2 | 652.70 | 713.49 | 694.04 | 15.32 |
| 24.7 | 1,749.70 | 1,425.67 | 1,728.65 | 77.87 |
| 74.1 | 5,047.60 | 3,379.31 | 4,014.35 | 26.98 |
| 222.2 | 13,545.06 | 8,288.73 | 9,401.24 | 132.49 |
| 666.7 | 23,777.41 | 13,864.18 | 16,945.12 | 407.83 |
| 2000 | 28,225.74 | 21,608.65 | 23,675.77 | −65.28 |
| 6000 | 28,991.06 | 21,786.48 | 24,932.36 | −33.28 |
| 18000 | 27,582.99 | 20,285.95 | 22,278.00 | 13.98 |

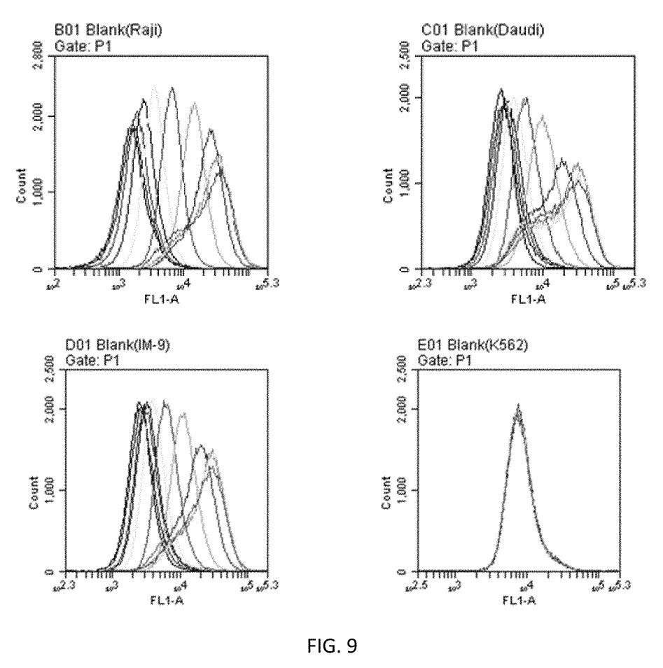

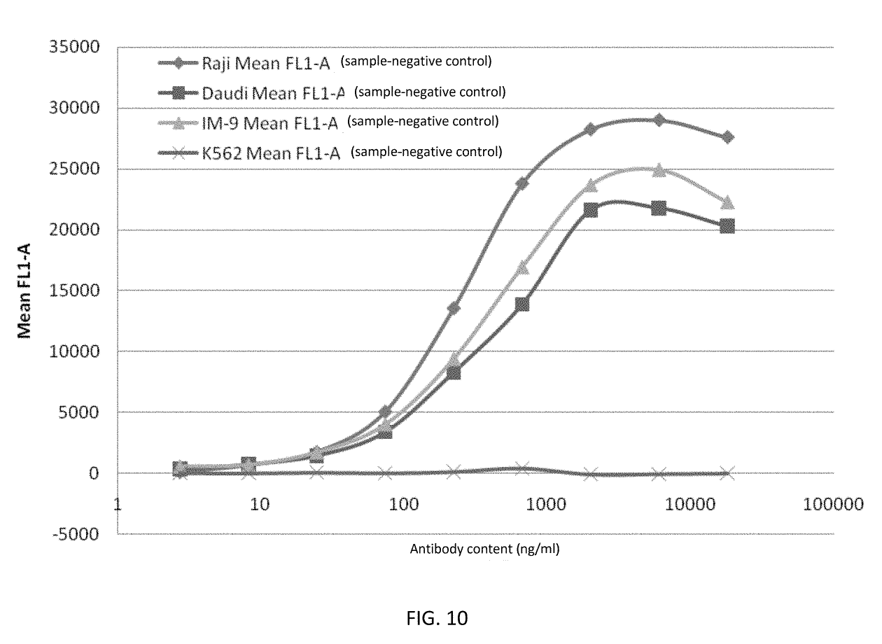

Except that the blank control well A10 was added with 50 μl of 0.02 mol/L PBS (pH 7.4, containing 1% BSA), the remaining wells were each added with 50 μl of well diluted mouse anti-human IgG K chain monoclonal antibody-FITC (1:1000), well mixed and reacted at room temperature for 30 min, and centrifuged, and then the supernatant was carefully pipetted and discarded. 150 μl of 0.02 mol/L PBS (pH 7.4) was added into each well, and the cells in the well were resuspended. The flow cytometer in-door sample loading quantity was set as 50,000 events, and the flow rate was set as Fast. The cells were well mixed by carefully pipetting up and down them with the tip of a pipette, the cell suspension was transferred into a 0.5 ml centrifuge tube, and the cell fluorescence values were determined sequentially. The results determined by the flow cytometer were shown in FIG. 9, and the reaction curve was shown in FIG. 10. By using the GraphPad Prism5.0 software, the EC₅₀ values of the K193 antibody binding to the Raji, Daudi and IM-9 cells were respectively 231.6, 359.9, 324.5 ng/ml, or 3.08×10⁻⁹ mole/L, 4.78×10⁻⁹ mole/L, 4.31×10⁻⁹ mole/L, and the K193 antibody has no binding activity to K562 cells.

2. Binding Activity Comparison Between K193 and Anti-CD19 Humanized Monoclonal Antibody K19

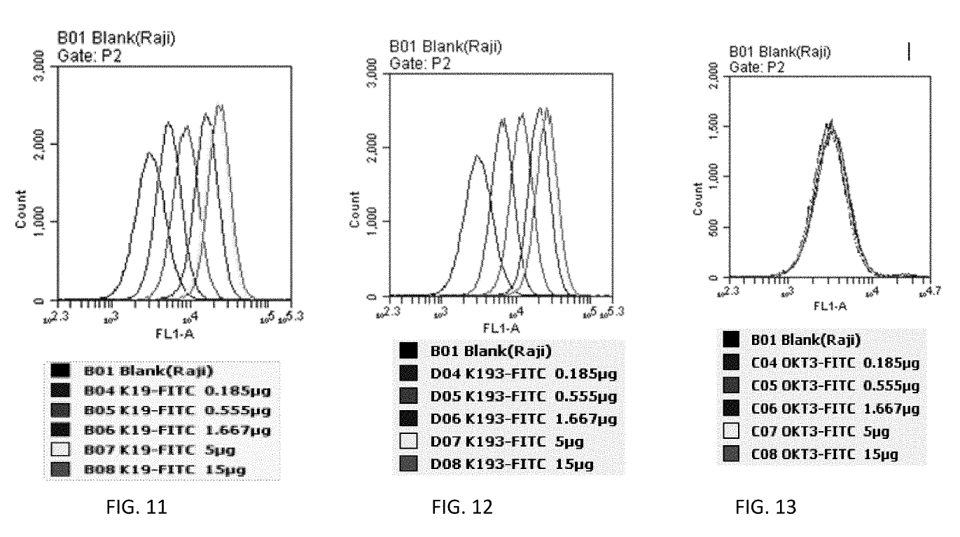

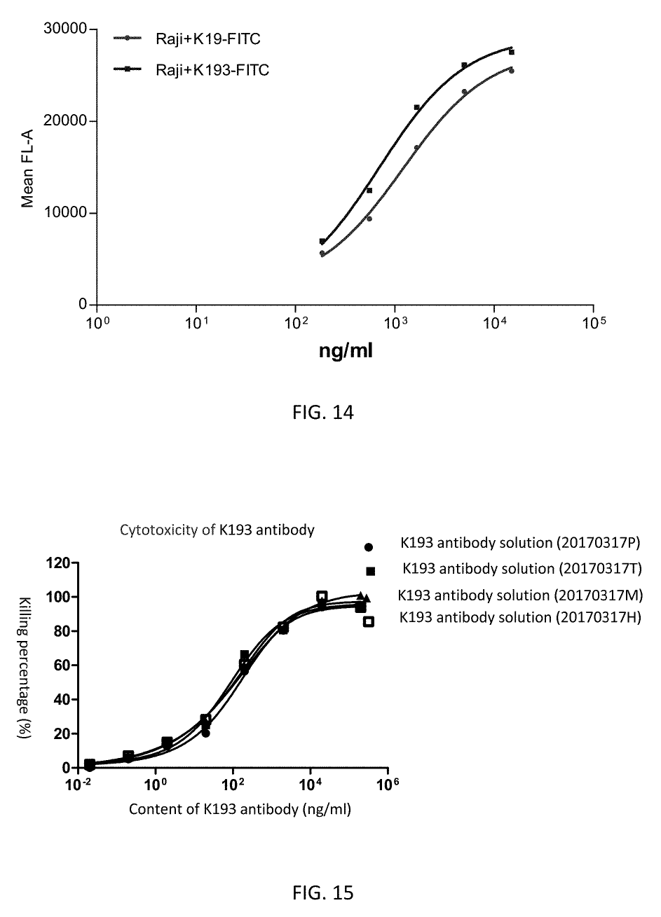

The specific binding activities of the K193 antibody and the anti-CD19 humanized monoclonal antibody K19 to Raji were detected with the flow cytometer (Accuri™ C6 Flow Cytometer, Becton Dickinson), and the test used the monoclonal antibody OKT3 as a control. Monoclonal antibodies K193-Biotin, K19-Biotin and OKT3-Biotin were diluted with 0.02 mol/L PBS (pH 7.4, containing 1% BSA) until the initial protein concentration was 15 μg/ml and the monoclonal antibodies were subjected to 3-fold series dilution to 0.185 μg/ml. A Raji cell suspension with a cell density of 5.0×10⁶ cells/ml was prepared, 100 μl of the cell suspension was added into each well, well mixed and reacted for 60 min. The flow cytometer was set at FAST for determining 50,000 events, and the average fluorescence value of cells in each well was sequentially determined. The determination results by the flow cytometer were shown in FIGS. 11-13 and the table below, and the dose response curve was shown in FIG. 14. By using the GraphPad Prism5.0 software, the EC50 values of the K19 monoclonal antibody and the K193 antibody binding to Raji cells were respectively calculated as 1,206 ng/ml and 697.2 ng/ml, or 8.06×10⁻⁹ mole/L and 9.25×10⁻⁹ mole/L. It can be seen from the calculation results that the binding activities of the K193 antibody and the monoclonal antibody K19 to a CD19 membrane antigen were highly consistent.

| Antibody Content | Raji + | Raji + | Raji + |
|---|---|---|---|
| (μg/ml) | K19-FITC | OKT3-FITC | K193-FITC |
| 0.185 | 5,659.39 | 3,860.22 | 6,966.38 |
| 0.556 | 9,370.61 | 3,392.86 | 12,475.99 |
| 1.667 | 17,131.86 | 3,566.70 | 21,510.99 |
| 5 | 23,221.22 | 3,460.83 | 26,112.11 |
| 15 | 25,462.85 | 3,708.05 | 27,511.30 |

### Embodiment 6: Bioactivity Determination of the CD19-CD3 Bispecific Antibody

This experiment determined the cytotoxic activity of the CD19-CD3 bispecific antibody by a cytotoxicity assay based on fluorescent dye release.

Well-grown Raji cells were pipetted up and down uniformly and then sampled for counting. According to the counting result and experimental requirements, a certain volume of the Raji cells was taken out and transferred into a centrifuge tube, centrifuged in a centrifugal machine at 800 rpm for 10 min, the supernatant was discarded, and the cells were washed with a HBSS solution 3 times. Then, 4 ml of the HBSS solution was added into to the centrifuge tube to suspend the Raji cells by pipetting up and down, 20 μl of a Fluo 3-AM stock solution (1 mmol/L, 5 μl of the Fluo 3-AM stock solution being added per 1 ml of the cell suspension) was added, and then 10 μl of 20% Pluronic F-127 (2.5 μl of the 20% Pluronic F-127 being added per 1 ml of the cell suspension) was added and well mixed, and was allowed to stand in an incubator at 37° C. for 60 min. Thereafter, the solution was centrifuged in the centrifugal machine at 800 rpm for 10 min, the supernatant was discarded, and the cells were washed with the HBSS solution 3 times to sufficiently remove the residual Fluo3-AM working solution, and then the Raji cells were adjusted to 4×10⁶ cells/ml using the HBSS solution.

Well-grown Jurkat cells (CD4+) were pipetted up and down uniformly and then sampled for counting. According to the counting result and experimental requirements, a certain volume of the Jurkat cells was taken out and transferred into a centrifuge tube, centrifuged in a centrifugal machine at 800 rpm for 10 min, the supernatant was discarded, and the cells were washed with a HBSS solution 3 times. The Jurkat cell density was then adjusted to 4×10⁷ cell/ml using the HBSS solution. The adjusted Raji cells and Jurkat cells were uniformly mixed in equal volume, and then added into respective wells in columns 2-10 of a 96-well all-black fluorescent plate by a micropipettor, with a dose of 100 μl/well.

One irradiated deep-well dilution plate was taken, and 4 batches of K193 antibody solutions (batch number: 20170317P, 20170317T, 20170317M, 20170317H) were subjected to 10 times dilution in the deep-well dilution plate between 200 ng/ml-0.02 pg/ml according to the labeled protein content, with 8 dilutions in total. The diluted 4 batches of K193 antibodies were then sequentially transferred into respective wells in columns 3-10 of the aforementioned 96-well all-black fluorescent plate (2 replicate wells per batch of K193 antibodies) using a multi-channel micropipettor, with a dose of 100 μl/well. The HBSS solution was added into the wells A2-D2 in column 2 of the 96-well all-black fluorescent plate with a dose of 100 μl/well, to serve as a blank control. The HBSS solution was added into the wells E2-H2 in column 2 of the 96-well all-black fluorescent plate with a dose of 95 μl/well, and then a 2% saponin solution was added into the wells with a dose of 5 μl/well to serve as a positive control. The 96-well all-black fluorescent plate was incubated in an incubator under 8% CO₂ at 37° C. for 4 h.

The switch of a TECAN multi-function microplate reader was turned on to select options for fluorescence intensity determination: the determination was started by setting the excitation wavelength at 488 nm, setting the emission wavelength at 526 nm, and selecting optimized options for the gain value. The cell kill rate was calculated according to the equation below:

Cell kill rate=(measurement of sample well−blank)/(measurement of positive control well−blank)×100%

The cytotoxic dose response curve of the K193 antibody was shown in FIG. 15. The results of the experiments showed that 4 batches of CD19-CD3 bispecific antibodies all can kill more than 50% of the B cell-derived tumor cells at the pg/ml level. The ED50s of the K193 antibodies of batches 20170317P, 20170317T, 20170317M and 20170317H in killing B lymphoma cells were sequentially 133.30, 83.60, 131.20, 97.84 pg/ml, and the corresponding molar concentrations were sequentially: 1.77×10⁻¹² mole/L, 1.11×10⁻¹² mole/L, 1.74×10⁻¹² mole/L, and 1.30×10⁻¹² mole/L. It could be seen from these results that the concentration at which the K193 antibody gives play to its function was very low.

Fluorescence values corresponding to respective concentrations of K193 antibodies in activating the T cell to kill B cells

| K193 Antibody | K193 antibody | | K193 antibody | | K193 antibody | | K193 antibody | |
|---|---|---|---|---|---|---|---|---|
| (pg/ml) | solution (20170317P) | | solution (20170317T) | | solution (20170317M) | | solution (20170317H) | |
| 200000 | 46923 | 44979 | 45754 | 46711 | 46807 | 49670 | 48030 | 47356 |
| 20000 | 45940 | 46152 | 46291 | 46970 | 47309 | 46423 | 50615 | 50891 |
| 2000 | 39599 | 40308 | 39311 | 40239 | 39862 | 38959 | 42887 | 41640 |
| 200 | 28932 | 29606 | 33381 | 33300 | 29517 | 30668 | 32798 | 31278 |
| 20 | 12986 | 13006 | 14214 | 15726 | 16280 | 15247 | 16823 | 16995 |
| 2 | 9471 | 9616 | 9922 | 10612 | 10605 | 10615 | 10775 | 10745 |
| 0.2 | 6236 | 6271 | 6641 | 6249 | 6771 | 7078 | 6938 | 7124 |
| 0.02 | 3821 | 4111 | 4274 | 4220 | 4355 | 4515 | 4766 | 4596 |
| Blank | 3934 | 3890 | 3783 | 3803 | 3787 | 3859 | 3734 | 3714 |
| PC | 48095 | 49383 | 49262 | 47508 | 47090 | 48682 | 49852 | 51250 |

The killing percentage was calculated after the fluorescence values were averaged.

The ED50 values calculated by using the GraphPad Prism 5 software were shown in the table below

| Content of | K193 antibody | K193 antibody | K193 antibody | K193 antibody |
|---|---|---|---|---|
| K193 antibody | solution | solution | solution | solution |
| (pg/ml) | (20170317P) | (20170317T) | (20170317M) | (20170317H) |
| 200000 | 93.8% | 95.2% | 100.8% | 93.9% |
| 20000 | 94.0% | 96.1% | 97.7% | 100.4% |
| 2000 | 80.4% | 80.7% | 80.8% | 82.3% |
| 200 | 56.6% | 66.3% | 59.6% | 60.5% |
| 20 | 20.3% | 25.1% | 27.1% | 28.2% |
| 2 | 12.6% | 14.5% | 15.4% | 15.0% |
| 0.2 | 5.2% | 5.9% | 7.0% | 7.1% |
| 0.02 | 0.1% | 1.0% | 1.4% | 2.0% |
| ED50 | 133.30 | 83.60 | 131.20 | 97.84 |
| (pg/ml) | | | | |

### Embodiment 7. Binding Activity of the CD19-CD3 Bispecific Antibody to an Extracellular Region of a Genetically Engineered Recombinant Human CD3ε

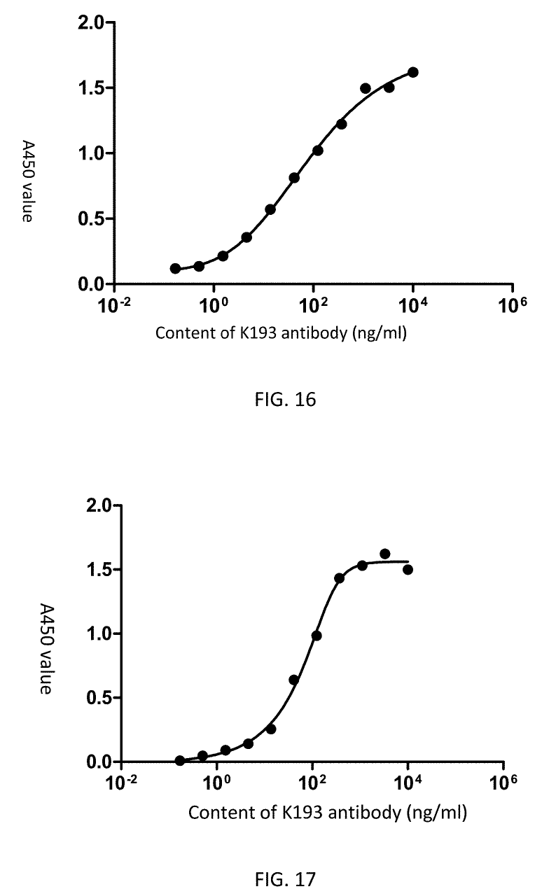

The recombinant human CD3 ε (Sinocelltech Ltd., batch number: LC11MA1103) was dissolved in 0.5 ml of water for injection, and then diluted with a carbonate coating buffer to 0.4 μg/ml, and coated onto a 96-well elisa plate (Shenzhen Jincanhua Industry Co. Ltd) with a dose of 100 μl per well, the coated elisa plate was placed at 37° C. for 2 h, and then placed in a refrigerator overnight at 2-8° C. The elisa plate was washed with a washing solution (20 mmol/L PBS-T, pH 7.4) 3 times and patted dry. Each well of the elisa plate was added with 220 μl of a blocking solution (20 mmol/L phosphate buffered saline with Tween 20 (PBS-T) containing 2% bovine serum albumin (BSA)), and then placed at 37° C. for 60 min. The elisa plate was washed with a washing solution 3 times and patted dry. The K193 antibody-Biotin was pre-diluted to 10 μg/ml according to a protein content by using 20 mmol/L PBS (pH 7.4), and the wells B2-C2 of the blocked elisa plate were added with the diluted K193 antibody-Biotin at a dose of 100 μl per well (2 wells per dilution), where the diluted K193 antibody-Biotin was subjected to 3 times serial dilution started from 10 μg/ml, with 11 dilutions in total; and the elisa plate added with samples was incubated at 37° C. for 60 min, then washed with a washing solution 4 times and patted dry. The diluted horseradish-peroxidase-labeled streptavidin-biotin (1:20,000) was added into each well of the elisa plate at a dose of 100 μl per well, and reacted through incubation at 37° C. for 60 min; and then the elisa plate was washed with a washing solution 5 times and patted dry. A Tetramethylbenzidine (TMB) developing solution was added into each well of the elisa plate at a dose of 100 μl per well, and reacted at 37° C. with protection from light for 15 min. 50 μl of a stop buffer (1 mol/L sulfuric acid) was added into each well to terminate the reaction. The A450 value was read using a Multiskan FC elisa plate. The A450 values corresponding to logarithms of respective concentrations of the K193 antibody were plotted as shown in FIG. 16. By using the GraphPad Prism 5 software and selecting a five-parameter curve equation, the EC₅₀ of the K193 antibody reaction=73.11 ng/ml, or 9.71×10⁻¹⁰ mole/L.

### Embodiment 8. Binding Activity of the CD19-CD3 Bispecific Antibody to an Extracellular Region of a Genetically Engineered Recombinant Human CD19

The recombinant human CD19 (Sinocelltech Ltd., batch number: LC10AU1901) was dissolved in 0.5 ml of distilled water, and then diluted with a carbonate coating buffer to 0.4 μg/ml, and coated onto a 96-well elisa plate with a dose of 100 μl per well, the coated elisa plate was placed at 37° C. for 2 h, and then placed in a refrigerator overnight at 2-8° C. The elisa plate was washed with a washing solution 3 times and patted dry. Each well of the elisa plate was added with 220 μl of the blocking solution (20 mmol/L PBS-T containing 2% BSA), and then placed at 37° C. for 60 min. The elisa plate was washed with a washing solution 3 times and patted dry. The K193 antibody-Biotin was pre-diluted to 10 μg/ml according to a protein content by using 20 mmol/L PBS (pH 7.4), and the wells B2-C2 of the blocked elisa plate were added with the diluted K193 antibody-Biotin at a dose of 100 μl per well (2 wells per dilution), where the diluted K193 antibody-Biotin was subjected to 3 times serial dilution started from 10 μg/ml, with 11 dilutions in total; and the elisa plate added with samples was incubated at 37° C. for 60 min, then washed with a washing solution 4 times and patted dry. The diluted horseradish-peroxidase-labeled biotin (1:8000) was added into each well of the elisa plate at a dose of 100 μl per well, and reacted through incubation at 37° C. for 60 min. Thereafter, the elisa plate was washed with a washing solution 5 times and patted dry. A TMB developing solution was added into each well of the elisa plate at a dose of 100 μl per well, and reacted at 37° C. with protection from light for 10-15 min. 50 μl of a stop buffer (1 mol/L sulfuric acid) was added into each well to terminate the reaction. The A450 value was read using the Multiskan FC elisa plate. The dose reaction curve of respective concentrations of the K193 antibody versus A450 values is shown in FIG. 17. By using the GraphPad Prism 5 software and selecting a five-parameter curve equation, the EC₅₀ of the K193 antibody reaction=70.65 ng/ml, or 9.38×10⁻¹⁰ mole/L.

### Embodiment 9. Determination of the Binding Activity of the CD19-CD3 Bispecific Antibody Through a Recombinant CD3-ε-and-CD19 Double Antigen Sandwich Method

The recombinant human CD3ε (Sinocelltech Ltd., batch number: LC11MA1103) was dissolved in 0.5 ml of water for injection, and then diluted with a carbonate coating buffer to 0.4 μg/ml, and coated onto a 96-well elisa plate (Shenzhen Jincanhua Industry Co. Ltd) with a dose of 100 μl per well, the coated elisa plate was placed at 37° C. for 2 h, and then placed in a refrigerator overnight at 2-8° C. The elisa plate was washed with a washing solution 3 times and patted dry. Each well of the elisa plate was added with 220 μl of the blocking solution (20 mmol/L PBS-T containing 2% BSA), and then placed at 37° C. for 60 min. The elisa plate was washed with a washing solution 3 times and patted dry. 3 batches of K193 antibody solutions (bath number: 20171001, 20171002, 20171003) were pre-diluted to 10 μg/ml according to a protein content by using 20 mmol/L PBS (pH 7.4), and the wells in column 1 of the blocked elisa plate were sequentially added with the diluted 3 batches of K193 antibody solutions at a dose of 100 μl per well (2 wells per dilution), where the diluted 3 batches of K193 antibody solutions were subjected 3 times to serial dilution started from 10 μg/ml, with 11 dilutions in total; and the elisa plate added with samples was incubated at 37° C. for 60 min, then washed with a washing solution 4 times and patted dry.

| | K193 antibody | K193 antibody | K193 antibody |
|---|---|---|---|
| Protein content | solution | solution | solution |
| (ng/ml) | 20171001 | 20171002 | 20171003 |
| 10000 | 2.3328 | 2.3775 | 2.4206 |
| 3333.333 | 2.3927 | 2.2643 | 2.5212 |
| 1111.111 | 2.3746 | 2.3455 | 2.5459 |
| 370.370 | 2.0384 | 2.0146 | 2.2328 |
| 123.457 | 1.5076 | 1.4575 | 1.6311 |
| 41.152 | 0.9622 | 0.9061 | 1.0034 |
| 13.717 | 0.5709 | 0.5173 | 0.5947 |
| 4.572 | 0.3842 | 0.3408 | 0.3756 |
| 1.524 | 0.3375 | 0.2686 | 0.2938 |
| 0.508 | 0.1974 | 0.2068 | 0.2137 |
| 0.169 | 0.2132 | 0.1972 | 0.1795 |
| EC₅₀ (ng/ml) | 82.45 | 87.32 | 76.66 |
| EC₅₀ (mole/L) | 1.09 × 10⁻⁹ | 1.16 × 10⁻⁹ | 1.02 × 10⁻⁹ |

The biotin-labeled recombinant human CD19 was diluted to 100 ng/ml, and was added into each well of the elisa plate at a dose of 100 μl per well. The reaction was conducted through incubation at 37° C. for 60 min, then washed with the washing solution for four times and patted dry. The diluted horseradish-peroxidase-labeled streptavidin-biotin (1:20000) was added into each well of the elisa plate at a dose of 100 μl per well, and reacted through incubation at 37° C. for 60 min. Thereafter, the elisa plate was washed with a washing solution 5 times and patted dry. A TMB developing solution was added into each well of the elisa plate at a dose of 100 μl per well, and reacted at 37° C. with protection from light for 10 min. 50 μl of a stop buffer (1 mol/L sulfuric acid) was added into each well to terminate the reaction. The A450 value was read using the Multiskan FC elisa plate. By using the GraphPad Prism 5 software and selecting a five-parameter curve equation, the EC₅₀ values of the K193 antibody solutions in the double-antigen sandwich euzymelinked immunosorbent assay reaction were calculated, where the EC₅₀ values of respective batches of the K193 antibodies were respectively 82.45, 87.32 and 76.66 ng/ml, or 1.09×10, 1.16×10, and 1.02×10⁻⁹ mole/L, showing that the respective batches are highly consistent.

### Embodiment 10. T Lymphocyte Proliferation Test of the CD19-CD3 Antibody

A CD69 molecule was an early marker of T cell activation, and Jurkat E6-1 cells cultured under normal conditions rarely expressed the CD69 molecule. Jurkat E6-1 cells and Raji cells were co-cultured in appropriate proportions, and in presence of CD3 molecule activators such as antibodies OKT3 and K193, CD69 could be expressed on the surfaces of the Jurkat E6-1 cells, and the expression level was positively correlated with the concentration of a stimulator; the OKT3 molecule (anti-CD3 monoclonal antibody) could better stimulate a T cell to produce the CD69 molecule only when it acted together with a second stimulating factor, and a single OKT3 molecule could also activate low-abundance expression of the CD69 molecule at a higher concentration.

### 1. Assay of T Cell Proliferation Stimulated by the K193 Antibody and the OKT3 Monoclonal Antibody in Presence of B Cells

Jurkat E6-1 cells cultured in 10% FCS 1640 culture solution were collected by centrifugation, subjected to adjustment of the cell concentration, and then mixed with Raji cells in such a manner that the mixed cell suspension contained 2×10⁶/ml of Jurkat E6-1 cells and 2×10⁵/ml of Raji cells. The aforementioned cell suspension was inoculated into a 24-well cell culture plate, added with serially-diluted K193 antibodies and OKT3 antibodies, then cultured in an incubator under 10% CO2 at 37° C. for 18 h, and centrifuged. The supernatant was removed, and then the solution was reacted with 5 μl of an Anti-Human CD4 FITC+Anti-Human CD69 PE (clone: FN50, LOT: E13987-103, eBioscience Anti-Human CD4 FITC, clone: OKT4, LOT: E10526-1634, eBioscience) mixed (1:1) sample with protection from light for 30 min, and then the cell expression of CD69 and CD4 was determined by the flow cytometer. The determined cell fluorescence levels were compared to observe the reaction relationship between the amount of CD69 expression after stimulation by respective concentrations of K193 or OKT3 (positive control). The Mean FL2-A values (CD69) corresponding to the two antibodies were processed by software analysis. The parameters in the table below were calculated using the GraphPad Prism 5.0 software. The used measuring unit of the antibodies was ng, and the logarithmic value thereof was taken for calculation, where the ED50 value corresponding to the K193 antibody was 0.08663 ng/ml, and the ED₅₀ value corresponding to the OKT3 antibody was 1,278 ng/ml, and thus a proportional relationship of about 1.47×10⁴ times existed between them. That is, in the presence of the B cells, the amount of CD69 expression as stimulated by the OKT3 was significantly lower than that of the K193 antibody.

| | Mean FL1-A(CD4) | | Mean FL2-A(CD69) | |
|---|---|---|---|---|
| Antibody Content | T + B(10:1) + OKT3 | T + B(10:1) + K193 | T + B(10:1) + OKT3 | T + B(10:1) + K193 |
| (ng/ml) | Antibody | Antibody | Antibody | Antibody |
| 0.00002 | / | 3,492.37 | / | 2,967.17 |
| 0.0002 | / | 3,477.21 | / | 3,132.86 |
| 0.002 | / | 3,440.59 | / | 3,193.65 |
| 0.02 | 3,667.01 | 3,267.57 | 3,113.05 | 5,925.08 |
| 0.2 | 3,594.62 | 2,967.52 | 2,590.94 | 8,875.99 |
| 2 | 3,592.47 | 2,851.32 | 2,773.25 | 12,386.15 |
| 20 | 3,600.96 | 2,697.68 | 2,898.15 | 9,241.00 |
| 200 | 3,523.06 | 2,730.23 | 3,349.41 | 8,073.21 |
| 2000 | 3,288.83 | / | 4,913.65 | / |
| 20000 | 3,095.80 | / | 6,084.64 | / |
| 200000 | 2,930.10 | / | 4,690.87 | / |

2. Activation of T Cells by the K193 Antibody Requires Synergistic Stimulation of B Cells

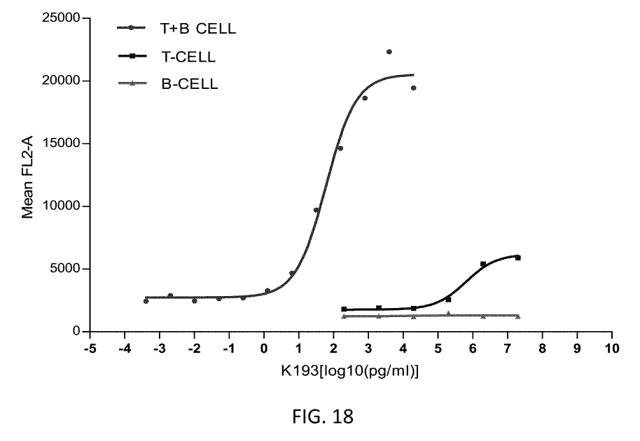

Jurkat E6-1 cells cultured in 10% FCS 1640 culture solution were collected by centrifugation, subjected to adjustment of the cell concentration until the cell concentration was 3×10⁶/ml, and then ready for use; (1) after the cells were mixed with the Raji cells, the cell suspension contains 2×10⁶/ml of Jurkat E6-1 cells and 2×105/ml of Raji cells; (2) Jurkat E6-1 cells with a cell concentration of 3×10⁶/ml were added into ⅓ volume of the cell culture solution; (3) the cell concentration of the Raji cells was 2×10⁵/ml; the aforementioned cell suspensions were inoculated into a 24-well cell culture plate, added with serially-diluted K193 antibodies, then cultured in an incubator under 10% CO2 at 37° C. for 18 h, and centrifuged, the supernatant was removed, and then the solution was reacted with 5 μl of an Anti-Human CD69 PE (clone: FN50, LOT: E13987-103, eBioscience) mixed (1:1) sample with protection from light for 30 min, and then the average fluorescence intensity of CD69 expressed in cells was determined by the flow cytometer. The results were shown in the table below. A graph was plotted by using the determined concentrations of K193 as the abscissa and using the average fluorescence intensity of the cells as the ordinate, as shown in FIG. 18, so as to observe the reaction relationship of the amount of CD69 expression after stimulation by respective concentrations of K193 (positive control).

| Content of | | | |
|---|---|---|---|
| K193 antibody | Mean FL2-A(CD69) | | |
| (pg/ml) | T + B CELL | T-CELL | B-CELL |
| 0.0004096 | 2431.57 | | |
| 0.002048 | 2887.72 | | |
| 0.01024 | 2443.84 | | |
| 0.0512 | 2644.63 | | |
| 0.256 | 2705.42 | | |
| 1.28 | 3274.41 | | |
| 6.4 | 4682.14 | | |
| 32 | 9699.76 | | |
| 160 | 14624.27 | | |
| 800 | 18628.34 | | |
| 4000 | 22321.99 | | |
| 20000 | 19447.03 | | |
| 200 | | 1,802.33 | 1,247.95 |
| 2000 | | 1,908.16 | 1,254.63 |
| 20000 | | 1,876.30 | 1,220.62 |
| 200000 | | 2,556.53 | 1,492.16 |
| 2000000 | | 5,412.77 | 1,247.13 |
| 20000000 | | 5,905.11 | 1,227.56 |
| EC50 | 60.95 | 642160 | — |

It could be seen from calculation of the above data that, the ED₅₀ value corresponding to a (T+B) Cell+K193 antibody group was 60.95 pg/ml, the ED₅₀ value corresponding to a T Cell+K193 antibody group was 642 ng/ml, and thus a proportional relationship of about 1.05×10⁴ times was existed between them. In the absence of B cells, the MFI of CD69 is lower, and even if the concentration of K193 reached 20 μg/ml, MFI could only reach the level of 5,900, about 3 times larger than the base background value, so that a single K193 could not stimulate activation of T cells well.

From the results of this experiment, it was known that Jurkat E6-1 cells activated by the K193 expressed a large amount of CD69 molecules on surfaces thereof, which was a co-stimulatory effect of the K193 antibody under the co-stimulation in the presence of Raji cells. These two conditions were indispensable and one of them could not achieve efficient activation of T cells. Under the premise of the presence of B cells, within a certain concentration range the amount of CD69 molecules produced by T cells was positively correlated with the amount of K193.

### Embodiment 11 Study on the Actions Mechanism of Stimulating T Cells by the K193 Antibody

It could be clearly seen from Embodiment 10 that in the presence of B cells, the K193 antibody could trigger T cell activation at a very low concentration, which is extremely inconsistent with activation of T cells by OKT3. This was probably because that the B7:CD28 costimulatory signal was involved in the activation process. CD80 (B7.1) and CD86 (B7.2) were molecules on the surface of B lymphocyte membrane, CD28 is a molecule on the surface of T lymphocyte membrane, and all of them were members of the costimulatory molecule immunoglobulin superfamily. B7 expressed on B cells or antigen presenting cells bound to CD28 expressed on T cells, and the co-stimulatory signal mediated by them was required for T cell activation, proliferation and effect production. CD86 interacted with CD28, and were major synergistic factors in inducing T lymphocyte proliferation and production of IL-2.

OKT3 was a mouse anti-human CD3 monoclonal antibody, and binding of high concentration of the monoclonal antibody OKT3 to CD3 ε on the surface of T cells could cause cross-linking of T cell TCR-CD3 complex and direct generation of a T cell activation signal without assistance of a second signal; and monoclonal antibody K19 could specifically bind to a CD19 site on the surface of a B cell. A monoclonal antibody K193 was a bispecific antibody that specifically bound to CD3 con the surface of human T cells and CD19 on the surface of B cells. Conducted studies have shown that the monoclonal antibody K193 could trigger T cell activation at a concentration below ng/ml in the presence of B cells. This Embodiment was intended to verify whether this ultra-efficient activation effect was enhanced by the B7:CD28 costimulatory molecule. It was designed that monoclonal antibodies against CD3 ε and CD19 were added into a reaction system to competitively and partially block the binding of K193 to CD19 and CD3 ε, and meanwhile monoclonal antibodies B7.1 (CD80) and B7.2 (CD86) were added to block the binding of B7 on the surface of B cells to the CD28 molecule on the surface of T cells, in order to prove by experiments that competitive blocking of these links could down-regulate expression of the CD69 and CD25 molecules.

The T lymphocytes cultured under normal conditions substantially did not express CD69 and CD25 molecules. The previous experiments showed that when a mixed culture of Jurkat E6-1 cells and Raji cells (T:B=10:1) was used for the experimental study, when the content of the monoclonal antibody K193 was in the range of 20 pg/ml-2000 pg/ml and T+B cells at this ratio was cultured in an incubator under 8% CO2 at 36.5° C., Jurkat E6-1 was subjected to co-stimulation by the monoclonal antibody K193 and B cells, CD69 molecules were abundantly expressed on the cell surface after 18 h of culture, and a large amount of CD25 molecules were produced after 40 h of culture. The concentration of the K193 antibody used in the present embodiment was 200 pg/ml, and the concentration of each of other monoclonal antibodies was selected to be 1 μg/ml. The antibodies used in this study were targeted to sites at CD80, CD86, CD28, CD3 and CD19, where anti-CD80 antibodies and anti-CD86 antibodies were rabbit monoclonal antibodies purchased from SinoCelltech Co., Ltd.; anti-CD19 monoclonal antibodies were humanized monoclonal antibodies prepared by our company, and anti-CD28 monoclonal antibodies and anti-CD3 monoclonal antibodies (OKT3) were mouse monoclonal antibodies purchased from Beijing Hongye innovative Antibody Technologies. The cells used were Raji and Jurkat E6-1 cells (ATCC, USA), the RPMI 1640 medium was purchased from Life Technologies Inc., USA, the fetal bovine serum was purchased from EXcell Biology Inc, and the cell culture plates were purchased from Nest Biotechnology Co., Ltd.

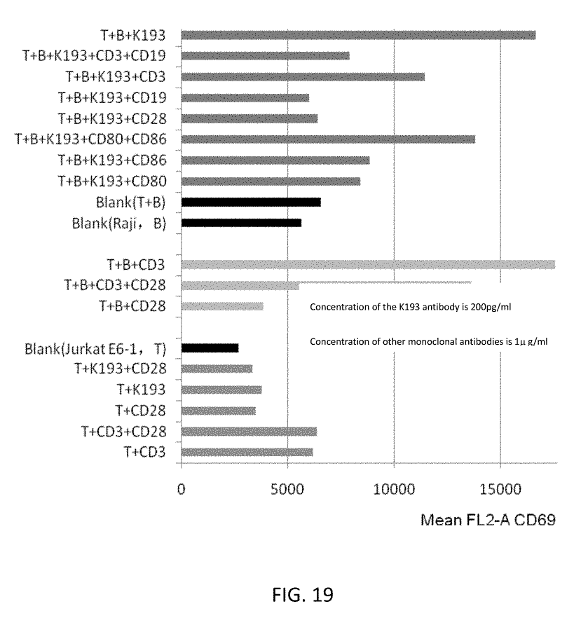

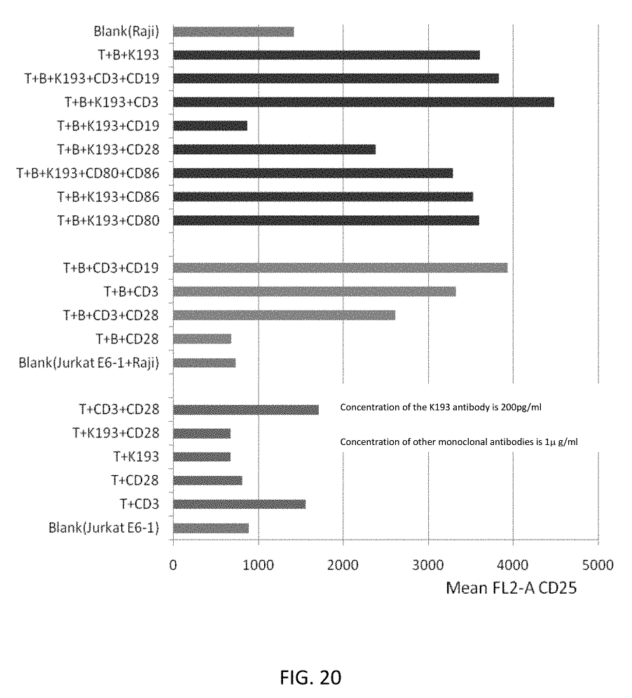

Monoclonal antibody reagents against CD80, CD86, CD28, K19, and OKT3 were taken out from the refrigerator at −20° C., and were placed at room temperature for melting, and the liquid in the bottle was gently shaken to mix the liquid thoroughly. The solutions of monoclonal antibodies against CD80, CD86, OKT3 and K19 were sequentially diluted until an antibody content was 8 μg/ml, and the anti-CD28 monoclonal antibody sample was diluted until the antibody content was 4 μg/ml. The K193 antibody was diluted to 800 pg/ml (the final concentration of the K193 antibody added into the 24-well plate was 200 pg/ml, and the final concentration of each of other monoclonal antibodies was 1 μg/ml), and the combination mode between antibodies was shown in FIG. 19.

Jurkat E6-1 cells and Raji cells were counted by a flow cytometer and centrifuged at 800 r/min cells to precipitate cells, the precipitated cells were resuspended in an appropriate volume of a cell culture medium until the concentrations of the Jurkat E6-1 cells (J) and Raji cells (B) were 3×10⁶ cells/ml and 3×10⁵ cells/ml, and the solutions were uniformly mixed in equal volume and added into respective wells of the 24-well plate at a dose of 600 μl/well. A suitable volume of the cell suspension was taken from the tube J and well mixed with an equal volume of 10% FCS in a RPMI 1640 cell culture medium to obtain a cell density of 1.5×10⁶ cells/ml. The cells were dispersed with a pipette, and added into a corresponding T cell blank control well of the 24-well plate at a dose of 600 μl. The cell culture plate was co-cultured in an incubator under 8% CO2 at 36.5° C. for 16-18 h. ½ volume of the cell culture in each well of the culture plate was taken out (the remaining cell culture was placed back into the incubator for continued culture for 40 h), centrifuged, added with anti-PE-labeled CD69 monoclonal antibody, and reacted for 60 min, then the average fluorescence intensity of cells in each well was detected by a BD C6 flow cytometer, and the results were determined one by one; the average intensity of CD expression in T cells as activated by each corresponding monoclonal antibody composition was shown in FIG. 19, and it could be seen from FIG. 19 that under the condition that T cells and B cells coexist, the existence of either one of OKT3 and K193 can stimulate T cells to produce high levels of CD69, and when other monoclonal antibodies were added into a T+B system, the amount of CD69 expressed by T cells was accordingly decreased, and the amount of CD69 expressed when OKT3 and K193 coexisted in the system was greater than that when either one of OKT3 and K193 existed, the large doses of anti-CD19 monoclonal antibodies and anti-CD28 monoclonal antibodies could significantly reduce the expression of CD69 in such a manner that the expression level of CD69 was equal to or lower than that of the blank control group. The coexistence of anti-CD80 monoclonal antibodies, anti-CD86 monoclonal antibodies and K193 also led to reduction in the expression level of CD69. When anti-CD80 monoclonal antibodies, anti-CD86 monoclonal antibodies and K193 antibodies existed simultaneously in the system, the expression amount of CD69 was relatively higher. The above results indicated that, although K193 could efficiently activate T cells in the presence of B cells, this activation could be terminated by adding ultra-high concentrations (the actual concentration of each monoclonal antibody in the system was 5,000 times larger than the concentration of K193) of anti-CD19 monoclonal antibodies and anti-CD28 monoclonal antibodies into the system, and could not be blocked by adding ultra-high concentrations of anti-CD80 monoclonal antibodies and anti-CD86 monoclonal antibodies into the system. Activation of T cells required co-stimulation of B cells, where the function of the CD28 costimulatory molecule was the most direct, but was far from enough, because high concentrations of anti-CD3 monoclonal antibodies and anti-CD28 monoclonal antibodies did not show greater superiority than pure anti-CD3 monoclonal antibodies in a system lacking B cells, and a low concentration of K193 antibodies had only a minor effect on activation of T cells.

The remaining cells in each well of the 24-well plate were cultured for 40 h, removed, centrifuged, added with PE-labeled anti-human CD25 monoclonal antibodies, reacted at 2-8° C. for 2 h, then the average fluorescence intensity of cells in each well was detected by the BD C6 flow cytometer, and the results were determined one by one; the average intensity of CD expression in T cells as activated by each corresponding monoclonal antibody composition was shown in FIG. 20. It could be seen from the figure that, under the condition that T cells and B cells coexist, the existence of either one of OKT3 and K193 can stimulate T cells to produce high levels of CD25, and when anti-CD19 monoclonal antibodies were added into a T+B system of K193, the amount of CD25 expressed by T cells was lower than that of a blank control group, and the amount of CD25 expressed when K193 and OKT3 coexisted in the system was significantly greater than that when either one of K193 and OKT3 existed, the anti-CD28 monoclonal antibodies could significantly reduce the expression of CD25, and the coexistence of anti-CD80 monoclonal antibodies, anti-CD86 monoclonal antibodies and K193 has no obvious effects on the expression of CD25. The above results indicated that, although K193 could efficiently activate T cells in the presence of B cells, this activation could be terminated by adding ultra-high concentrations (the actual concentration of each monoclonal antibody in the system was 5,000 times larger than the concentration of K193) of anti-CD19 monoclonal antibodies into the system; activation of T cells required co-stimulation of B cells, where the function of the CD28 costimulatory molecule has a certain effects, but the effects were far from enough, and high concentrations of anti-CD3 monoclonal antibodies and anti-CD28 monoclonal antibodies did not show greater superiority than pure anti-CD3 monoclonal antibodies in a system lacking B cells.

### Embodiment 12. Bispecific Antibody K193 Activates Human PBMC to Kill B Lymphoma Cells

Well-grown K562, Daudi, Namalwa and Raji cells were pipetted up and down uniformly and then sampled for counting. According to the counting result and experimental requirements, a certain volume of the cells was taken out and transferred into a centrifuge tube, centrifuged in a centrifugal machine at 800 rpm for 10 min, the supernatant was discarded, and the cells were washed with HBSS (Hank's balanced salt solution) 3 times. Then, 4 ml of the HBSS solution was added into to the centrifuge tube to suspend the cells by pipetting up and down, 20 μl of a Fluo 3-AM stock solution (1 mmol/L, 5 μl of the Fluo 3-AM stock solution being added per 1 ml of the cell suspension) was added, and then 10 μl of 20% Pluronic F-127 (in principle 2.5 μl of the 20% Pluronic F-127 being added per 1 ml of the cell suspension) was added and well mixed, and was allowed to stand in an incubator at 37° C. for 60 min. Thereafter, the solution was centrifuged in the centrifugal machine at 800 rpm for 10 min, the supernatant was discarded, and the cells were washed with the HBSS 3 times to sufficiently remove the residual Fluo3-AM working solution, and then the cells were adjusted to 4×10⁶ cells/ml using the HBSS.

The lymphocytes (PBMC) freshly isolated from the peripheral blood of a healthy person were sampled and counted. According to the counting result, an appropriate volume of human peripheral blood mononuclear cells (PBMC) were taken into a 15 ml centrifuge tube, centrifuged in a centrifugal machine at 800 rpm for 10 min, and the supernatant was discarded. Then the cells were washed twice with the HBSS. The PBMC density was then adjusted to 4×10⁷ cell/ml using the HBSS. The adjusted K562, Daudi and Namalwa cells were respectively well mixed with the human PBMC in equal volume, and then added into respective wells in columns 2-11 of a 96-well all-black fluorescent plate by a micropipettor, with a dose of 100 μl/well.

One irradiated deep-well dilution plate was taken, and the K193 antibody solution (stored at 2-8° C.) and BLI-193 (tandem single chain antibody structure, blinatumomab, −70° C.) were subjected to 10 times dilution in the deep-well dilution plate between 100 ng/ml-0.1 pg/ml according to the labeled protein content, with 7 dilutions in total. The diluted K193 antibodies were then sequentially transferred into respective wells of the aforementioned 96-well all-black fluorescent plate using a multi-channel micropipettor, with a dose of 100 μl/well. The HBSS solution blank controls were set in 6 wells with a dose of 100 μl/well. Into positive control wells of the 96-well plate added were 95 μl/well of the HBSS and 5 μl/well of a 2% saponin solution, and the 96-well all-black fluorescent plate was incubated in an incubator under 8% CO2 at 37° C. for 4 h.

The switch of a Bio-Tek multi-function microplate reader was turned on, and the determination was started by setting the excitation wavelength at 488 nm, setting the emission wavelength at 526 nm, and selecting optimized options for the gain value. The % cell kill was calculated according to the equation below:

Cell kill rate=(measurement of sample well−blank)/(measurement of positive control well−blank)×100%

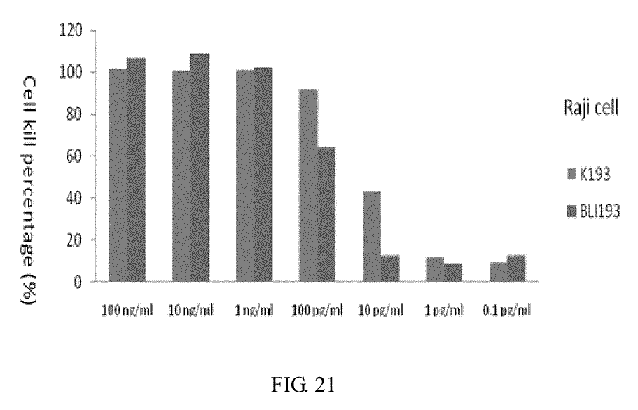

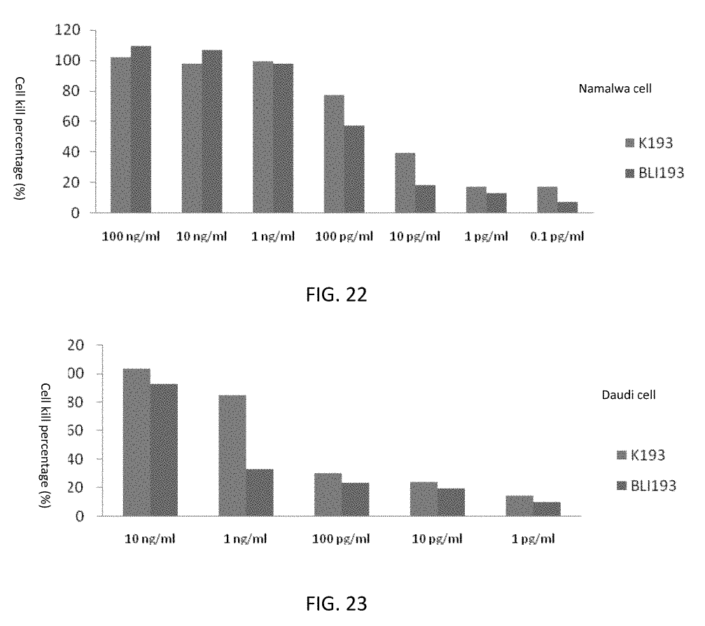

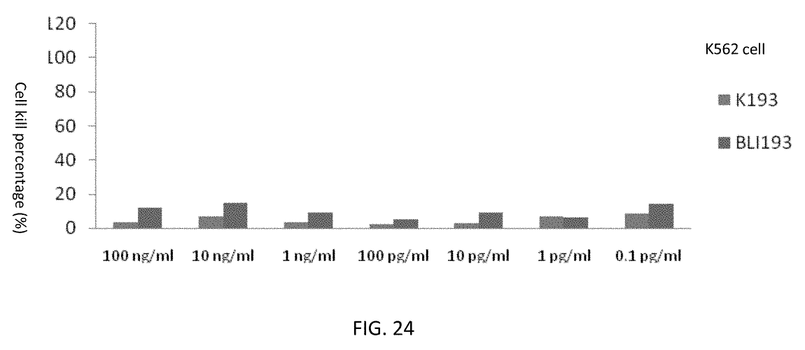

FIGS. 21-24 show the killing effect of human peripheral blood PBMC on CD19-positive and CD19-negative cells. FIGS. 21-24 show the killing effect of PBMC mediated by a bispecific antibody CD19-CD3 on CD19 positive cells.

K562 was a cell that did not express CD19 membrane antigen. It could be seen from the results that, K193 and BL1193 had almost no killing effect on it, and although respective concentrations of them showed a slight killing effect, the killing rate was very low and was independent of the concentration of the bifunctional antibody. K193 and BL1193 had strong killing effects on CD19-positive cells, and especially the killing effect of K193 was better than that of BL1193. By using the Graph Pad Prism 5.0 software, the ED₅₀ of the K193 antibody in killing Daudi, Namalwa and Raji cells were calculated as 410.3 pg/ml, 31.25 pg/ml, and 15.47 pg/ml, respectively; and the ED₅₀ of BLI193 in killing Daudi, Namalwa and Raji cells were 2,574.0 pg/ml, 107.4 pg/ml, and 86.80 pg/ml, respectively.

### ACRONYM AND ABBREVIATION LIST

μF microfarads
μg micrograms
μl microliter
μm micrometer
μM micromolar
° C. degrees Celsius
ADCC antibody-dependent cellular cytotoxicity
ALL acute lymphoblastic leukemia
BiTE Bispecific T cell Engager
blinatumomab MT103
B-NHLs B lymphocyte non-Hodgkin's lymphomas
BSA bovine serum albumin
CAR-T chimeric antigen receptor T cells
CDC complement dependent cytotoxicity
CHO Chinese hamster ovary
CLL chronic lymphocytic leukemia
cm centimeter
CR2 complement receptor 2
CTL cytotoxic T lymphocyte
EpCAM Epithelial cell adhesion molecule
Fab antigen-binding
h hour
HAMA human anti-mouse antibody
HBSS Hank's balanced salt solution
His histidine
His-Tag polyhistidine tail

### HPLC High Performance Liquid Chromatography

Hz hertz

### Ig Immunoglobulin

### IL-2 Interleukin-2

kDa kilodalton
kV kilovolt
L liter
m mass
MALT mucosa-associated lymphoid tissue lymphoma
MCL mantle cell lymphoma
mg milligram
MHC Major histocompatibility complex
min minute
ml/mL milliliter
mm millimeter
mM millimolar
mmol millimole
mol mole
mSiemens millisiemens
ng nanogram
NHL non-Hodgkin's lymphoma
nm nanometer
PBMC peripheral blood mononuclear cell
PBS phosphate buffer saline
PBS-T phosphate buffered saline with Tween 20
pg picogram
r revolutions
rpm rotations per minute
s second
ScFv single-chain variable fragment
SDS-PAGE sodium dodecyl sulphate-polyacrylamide gel electrophoresis
TAPA-1 target of the antiproliferative antibody
TCR T cell receptor
TCR T lymphocyte receptor

### TMB Tetramethylbenzidine

### U.S. FDA United States Food & Drug Administration

V volt
WM Waldenstrom's macroglobulinemias
z charge

What is claimed is:

1. A bispecific antibody that binds to human CD19 and CD3, wherein the bispecific antibody is composed of a Fab fragment which specifically recognizes a cell membrane antigen and a single-chain antibody which recognizes a CD3 molecule, wherein the single-chain antibody which recognizes the CD3 molecule is linked to a C-terminus of a CH1-region peptide fragment of the Fab fragment through a hydrophilic linker peptide-linker;
wherein the Fab fragment which specifically recognizes the cell membrane antigen contains a Fab structure which specifically recognizes a human CD19 antigen, and the bispecific antibody has the following structure: 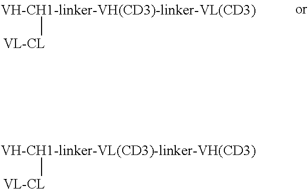
wherein the linker peptide-linker is composed of 8-20 hydrophilic amino acids.

2. The bispecific antibody according to claim 1, wherein the bispecific antibody has a structure as follows: 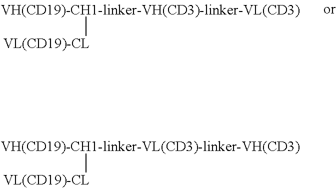
wherein the linker peptide-linker is a 2-3 fold polypeptide of the GGGGS form as a linker peptide.

3. The bispecific antibody according to claim 1, wherein the single-chain antibody which recognizes the CD3 molecule has a ScFv form structure, is targeted to human CD3ε, and can be derived from variable region gene sequences of various monoclonal antibodies currently known, comprising but not limited to CD3-specific antibodies OKT3, X35-3, WT31, WT32, SPv-T3b, TR-66, 11D8, 12F6, M-T301, SMC2 and F101.01.

4. The bispecific antibody according to claim 1, wherein the bispecific antibody has a structure as follows: 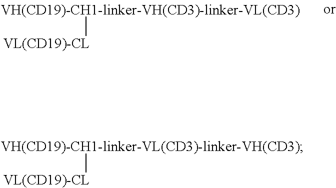
wherein the linker peptide-linker is a 2-3 fold polypeptide of the GGGGS form as a linker peptide;
wherein the Fab structural fragment specifically recognizing the human CD19 antigen can be derived from sequences of light chain variable regions and heavy chain variable regions of various well-known murine anti-human CD19 monoclonal antibodies, such as 4G7, B43, CLB-CD19, SJ25-C1, Leu-12, HD37 or other known variable region sequences of a monoclonal antibody against human CD19, or sequences of light chain variable regions and heavy chain variable regions of a monoclonal antibody against human CD19 constructed by our company, Beijing Luzhu Biotechnology Co., Ltd.

5. The bispecific antibody according to claim 2, wherein the bispecific antibody contains a heavy chain and a light chain each containing a nucleotide sequence and an amino acid sequence; wherein the bispecific antibody has a leader peptide on the heavy chain and a leader peptide on the light chain; wherein, the nucleotide sequence and amino acid sequence contained in the heavy chain containing the leader peptide are shown as sequences 1, 2, 4, and 5; the nucleotide sequence and amino acid sequence contained in the light chain containing the leader peptide are shown as sequences 7 and 8; the amino acid sequences contained in the heavy chain not containing the leader peptide are shown as sequence 3 and 6; and the amino acid sequence contained in the light chain not containing the leader peptide is shown as sequence 9.

6. A method for preparing the bispecific antibody of claim 1, wherein the bispecific antibody is prepared by a genetic recombination technology, and can be expressed in a CHO cell using various forms of mammalian cell expression vectors, preferably using a GS expression system, CHO cells are cultured using a chemically defined medium, and no hormones or proteins of various animal origins or hydrolyzates thereof are added during the culture.

7. A method for preparing the bispecific antibody according to claim 2, wherein the bispecific antibody is prepared by a genetic recombination technology, and can be expressed in a CHO cell using various forms of mammalian cell expression vectors, preferably using a GS expression system, CHO cells are cultured using a chemically defined medium, and no hormones or proteins of various animal origins or hydrolyzates thereof are added during the culture.

8. The preparation method according to claim 6, comprising linearizing a single plasmid vector containing a bispecific antibody gene by single endonuclease digestion; transfecting the linearized plasmid vector into a CHO cell to obtain a positive clone strain; culturing the positive clone strain in a bioreactor, such that a product is secreted into a supernatant of a culture solution; purifying by an ion exchange chromatography medium or affinity chromatography combined with ion exchange chromatography to obtain a bispecific antibody which can specifically bind to human CD19 and CD3.

9. The preparation method according to claim 7, comprising linearizing a single plasmid vector containing a bispecific antibody gene by single endonuclease digestion; transfecting the linearized plasmid vector into a CHO cell to obtain a positive clone strain; culturing the positive clone strain in a bioreactor, such that a product is secreted into a supernatant of a culture solution; purifying by an ion exchange chromatography medium or affinity chromatography combined with ion exchange chromatography to obtain a bispecific antibody which can specifically bind to human CD19 and CD3.

10. Use of the bispecific antibody according to claim 1 in preparation of drugs for treating various human-B-cell-derived malignant tumors or immune disorders such as various B cell leukemias (lymphomas), non-Hodgkin's lymphomas, and serious autoimmune diseases such as rheumatoid arthritis and ankylosing spondylitis.

11. Use of the bispecific antibody according to claim 2 in preparation of drugs for treating various human-B-cell-derived malignant tumors or immune disorders such as various B cell leukemias (lymphomas), non-Hodgkin's lymphomas, and serious autoimmune diseases such as rheumatoid arthritis and ankylosing spondylitis.

12. A pharmaceutical composition comprising the bispecific antibody according to claim 1.

13. A pharmaceutical composition comprising the bispecific antibody according to claim 2.

14. The pharmaceutical composition according to claim 12, wherein the pharmaceutical composition may be prepared into a liquid preparation or a freeze-dried preparation, and may be continuously administered by using a continuous infusion pump; or may be administered by a pulsed infusion pump at a fixed time, wherein intravenous administration is recommended; or may be administered by subcutaneous injection.

15. The pharmaceutical composition according to claim 13, wherein the pharmaceutical composition may be prepared into a liquid preparation or a freeze-dried preparation, and may be continuously administered by using a continuous infusion pump; or may be administered by a pulsed infusion pump at a fixed time, wherein intravenous administration is recommended; or may be administered by subcutaneous injection.


SEQUENCE LISTING (15 sequences)

SEQ ID NO: 1 - DNA, 1546 bp, Artificial Sequence
  Synthetic
aattgccgccaccatggaatggagctgggtgttcctgttctttctgtccgtgaccacaggcgtgcattctcaggtgcagctgcagcagtccggagctgaactggtgagacccggctccagcgtcaaaatttcctgtaaggctagcggatatgcattttctagttactggatgaattgggtgaagcagcgacctggacagggtctggagtggatcggccagatttggccaggcgatggagacaccaactacaatgggaagttcaaaggcaaggccaccctgacagctgacgaatcatccagcacagcatatatgcagctgtctagtctggcaagcgaggattctgccgtgtacttttgtgctaggcgggaaaccacaactgtcggcagatactattacgctatggactactgggggcagggaacaactgtgaccgtgagcagcgcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtagcggtggaggcggttcaggcggaggtggatccggcggtggcggcagcgaggtgcagctggtcgagtctggaggaggattggtgcagcctggagggtcattgaaactctcatgtgcagcctctggattcaccttcaataagtacgccatgaactgggtccgccaggctccaggaaagggtttggaatgggttgctcgcataagaagtaaatataataattatgcaacatattatgccgattcagtgaaagacaggttcaccatctccagagatgattcaaaaaacactgcctatctacaaatgaacaacttgaaaactgaggacactgccgtgtactactgtgtgagacatgggaacttcggtaatagctacatatcctactgggcttactggggccaagggactctggtcaccgtctcctcaggtggtggtggttctggcggcggcggctccggtggtggtggttctcagactgttgtgactcaggaaccttcactcaccgtatcacctggtggaacagtcacactcacttgtggctcctcgactggggctgttacatctggcaactacccaaactgggtccaacaaaaaccaggtcaggcaccccgtggtctaataggtgggactaagttcctcgcccccggtactcctgccagattctcaggctccctgcttggaggcaaggctgccctcaccctctcaggggtacagccagaggatgaggcagaatattactgtgttctatggtacagcaaccgctgggtgttcggtggaggaaccaaactgactgtccta

SEQ ID NO: 2 - protein, 511 aa, Artificial Sequence
  Synthetic
MEWSWVFLFFLSVTTGVHSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCSGGGGSGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 3 - protein, 492 aa, Artificial Sequence
  Synthetic
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCSGGGGSGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 4 - DNA, 1546 bp, Artificial Sequence
  Synthetic
aattgccgccaccatggaatggagctgggtgttcctgttctttctgtccgtgaccacaggcgtgcattctcaggtgcagctgcagcagtccggagctgaactggtgagacccggctccagcgtcaaaatttcctgtaaggctagcggatatgcattttctagttactggatgaattgggtgaagcagcgacctggacagggtctggagtggatcggccagatttggccaggcgatggagacaccaactacaatgggaagttcaaaggcaaggccaccctgacagctgacgaatcatccagcacagcatatatgcagctgtctagtctggcaagcgaggattctgccgtgtacttttgtgctaggcgggaaaccacaactgtcggcagatactattacgctatggactactgggggcagggaacaactgtgaccgtgagcagcgcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtagcggtggaggcggttcaggcggaggtggatccggcggtggcggcagccagactgttgtgactcaggaaccttcactcaccgtatcacctggtggaacagtcacactcacttgtggctcctcgactggggctgttacatctggcaactacccaaactgggtccaacaaaaaccaggtcaggcaccccgtggtctaataggtgggactaagttcctcgcccccggtactcctgccagattctcaggctccctgcttggaggcaaggctgccctcaccctctcaggggtacagccagaggatgaggcagaatattactgtgttctatggtacagcaaccgctgggtgttcggtggaggaaccaaactgactgtcctaggtggtggtggttctggcggcggcggctccggtggtggtggttctgaggtgcagctggtcgagtctggaggaggattggtgcagcctggagggtcattgaaactctcatgtgcagcctctggattcaccttcaataagtacgccatgaactgggtccgccaggctccaggaaagggtttggaatgggttgctcgcataagaagtaaatataataattatgcaacatattatgccgattcagtgaaagacaggttcaccatctccagagatgattcaaaaaacactgcctatctacaaatgaacaacttgaaaactgaggacactgccgtgtactactgtgtgagacatgggaacttcggtaatagctacatatcctactgggcttactggggccaagggactctggtcaccgtctcctca

SEQ ID NO: 5 - protein, 511 aa, Artificial Sequence
  Synthetic
MEWSWVFLFFLSVTTGVHSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 6 - protein, 492 aa, Artificial Sequence
  Synthetic
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 7 - DNA, 730 bp, Artificial Sequence
  Synthetic
aattgccgccaccatgtctgtgcctacccaggtgctgggactgctgctgctgtggctgacagacgcccgctgtgacatccagctgacacagtcaccagcatccctggccgtgagcctgggacagcgagcaactatctcttgcaaagcctcacagtccgtcgactatgatggggacagctatctgaactggtaccagcagatcccaggacagccccctaagctgctgatctacgacgctagtaatctggtgtcaggaatcccacccaggttcagcggttctggcagtggaactgattttaccctgaacattcaccccgtggagaaagtcgacgccgctacctatcattgccagcagtccacagaggacccctggactttcggcggagggaccaaactggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtagc

SEQ ID NO: 8 - protein, 239 aa, Artificial Sequence
  Synthetic
MSVPTQVLGLLLLWLTDARCDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECS

SEQ ID NO: 9 - protein, 219 aa, Artificial Sequence
  Synthetic
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECS

SEQ ID NO: 10 - DNA, 58 bp, Artificial Sequence
  Synthetic
cccaagcttaattgccgccaccatggaatggagctgggtgttcctgttctttctgtcc

SEQ ID NO: 11 - DNA, 57 bp, Artificial Sequence
  Synthetic
ttcctgttctttctgtccgtgaccacaggcgtgcattctcaggtgcagctgcagcag

SEQ ID NO: 12 - DNA, 25 bp, Artificial Sequence
  Synthetic
cgccaccgccggatccacctccgcc

SEQ ID NO: 13 - DNA, 58 bp, Artificial Sequence
  Synthetic
cccaagcttaattgccgccaccatgtctgtgcctacccaggtgctgggactgctgctg

SEQ ID NO: 14 - DNA, 58 bp, Artificial Sequence
  Synthetic
ctgggactgctgctgctgtggctgacagacgcccgctgtgacatccagctgacacagt

SEQ ID NO: 15 - DNA, 31 bp, Artificial Sequence
  Synthetic
ccggaattctcattagctacactctcccctg